(19) United States
(12) Patent Application Publication
(10) Pub. No.: US 2026/0148813 A1
(43) Pub. Date: May 28, 2026

(54) FORECASTING OF SUBJECT-RELATED ATTRIBUTES USING GENERATIVE MACHINE-LEARNING MODELS

(71) Applicants: HELMHOLZ ZENTRUM MÜNCHEN, München (DE); HOFFMANN-LA ROCHE INC., Little Falls, NJ (US)

(72) Inventors: Maria Bordukova, München (DE); Nikita Alexandrovich MAKAROV, München (DE); Michale P. Menden, Melbourne (AU); Raul Rodriguez-Esteban, Basel (CH); Fabian Schmich, Bad Feilnbach (DE)

(21) Appl. No.: 19/454,131

(22) Filed: Jan. 20, 2026

Related U.S. Application Data
(63) Continuation of application No. PCT/EP2024/070632, filed on Jul. 19, 2024.

(30) Foreign Application Priority Data
Jul. 21, 2023 (EP) 23187045.2

(51) Int. Cl. G16H 10/20; G06N 3/09; G16H 10/60; G16H 20/00

(52) U.S. Cl. CPC G16H 10/20; G06N 3/09; G16H 10/60; G16H 20/00

(57) ABSTRACT

A computer-implemented method of predicting, simulating, or forecasting values of one or more specified subject-related attributes during a clinical trial comprises: receiving input data comprising: a medical history of a subject, the medical history comprising values of a plurality of subject-related attributes of a subject; and data specifying a requested output, the data comprising: the one or more specified subject-related attributes of the subject and a time frame; and applying a trained generative machine-learning model to the received input data, the trained generative machine-learning model configured to generate output data based on the input data, the output data comprising: respective values of the one or more specified subject-related attributes of the subject in the specified time frame.

### CROSS REFERENCE TO RELATED APPLICATIONS

This application is a continuation of International Application No. PCT/EP2024/070632, filed internationally on Jul. 19, 2024, which claims priority to European Patent Application No. 23187045.2, filed on Jul. 21, 2023.

### TECHNICAL FIELD OF THE INVENTION

The present invention relates to a computer-implemented method of predicting, simulating, or forecasting values of one or more specified subject-related attributes during a clinical trial, or of determining an efficacy and/or safety of a therapeutic intervention during a clinical trial.

### BACKGROUND TO THE INVENTION

Only one out of ten compounds entering clinical trials will achieve regulatory approval [1]. The aim of clinical trials is to determine, as early as possible, the efficacy and safety of a compound based on the enrolled patients' data [2]. However, with around 80% of all trials being delayed due to patient enrolment [3], reducing the number of patients required to timely assess a compound is of utmost importance to accelerate drug development with a lower economic and societal burden.

AI progressively interacts with human intelligence and expert domain knowledge to support decision making in drug development [13]. In particular, machine learning (ML), a subfield of AI involving algorithms that learn from data, is increasingly being adopted in the field.

Consequently, interest in the application of ML to designing, conducting and analysing clinical trials has grown.

Artificial neural networks (NNs) are ML algorithms inspired by the structure of the human brain. NNs process the input signal through neurons organized in layers. The layers between the input and output are referred to as hidden layers, perform non-linear data transformations and are the key component that turns NNs into a powerful algorithm for data-driven modelling. Conventional ML methods, such as logistic regression or decision trees, typically require dimensionality reduction or manual feature selection, whereas NNs can directly process high-dimensional data and intrinsically learn feature representations. Besides that, NNs have been shown to be well suited for complex, multimodal, multidimensional and longitudinal data and have thus spearheaded developments in the field of digital twins (FIG. 9, panel a).

Conventional discriminative models learn the mapping between input and output data using regression or classification algorithms (FIG. 9, panel b), whilst generative models learn the distribution and sequential or temporal relations of the underlying data (FIG. 9, panel c). Generative models are able to produce synthetic data samples that are statistically similar to observed data. The data used to train patient-derived generative models can comprise data types, such as patient baseline measurements as well as prior clinical trajectories, consisting of endpoints, vitals, lab values and diagnoses taken at different time points (FIG. 9, panel a). As a result, such generative models can be initialized with real patient characteristics at a specific time point t and then simulate virtual patient trajectories starting at time point t+1, by sampling from the learned data distribution and sequential or time-dependent patterns (FIG. 9, panel c). We refer to these models as generative digital twins.

The company Unlearn. AI pioneered one of the first digital twins for clinical trials using generative NNs based on conditional restricted Boltzmann machines (CRBM; FIG. 9, panel e) [16, 17]. They leveraged data from placebo control arms of historical clinical trials and observational studies to train generative models that simulated patient trajectories for Alzheimer's disease [16] and multiple sclerosis [17]. A disadvantage of CRBMs is that they are shallow NNs containing a single hidden layer, which have a limited feature learning capability. For enhancing the quality of generated patient trajectories, modern NN architectures with multiple hidden layers can be used, which are denoted as deep NNs or deep learning.

Most of the recent advances in generative AI are being achieved by deep learning models. In the context of digital twins, a variational autoencoder (VAE) for stroke patient trajectory prediction was explored (FIG. 9, panel f) by Angiel et al., They leveraged EHR data to simulate trajectories of stroke patients in the treatment arm for the counterfactual scenario of placebo treatment. Using a VAE, patient trajectories were sequentially generated by decoding data sampled from a learnt low-dimensional embedding space of trajectories.

Current generative digital twin models for clinical trials exhibit limitations that reduce their applicability and generalizability. First, most efforts are limited to a single target use case of creating a digital twin-based control arm, whereby each enrolled patient in the treatment arm has a digital twin counterpart. Secondly, most methods rely on less than five thousand patients for training, which is considered small for deep learning [19], and thus may reduce the generalizability of the models. And, finally, the validation of digital twins is mostly based on statistical indistinguishability computed with statistical tests or by showing that linear or non-linear classifiers cannot distinguish between real patients and digital twins [16-18]. Only in exceptional cases was additional clinical data leveraged for validation, e.g. digital twins of multiple sclerosis.

Existing digital twin models in clinical trials do not use modern deep learning architectures yet. For instance, generative adversarial networks (GANs; FIG. 9, panel g) were successfully employed in a related field, i.e. simulating synthetic participants of a clinical trial that statistically replace patients actually enrolled into the trial to preserve privacy while enabling the sharing of data [21]. These synthetic entities cannot be considered digital twins as they do not simulate patient specific processes, but the approach could be potentially adapted for digital twins in the future. Modern generative deep learning models have the potential to implement more complex digital twins in clinical trials, such as diffusion models, which are state-of-the-art in image generation (FIG. 9, panel h); transformers, which have revolutionized language and speech generation (FIG. 9, panel i) [11], and neural ordinary differential equations, which enable learning of continuous dynamic systems (ODEs; FIG. 9, panel j) [12].

In summary, it has been observed that digital twins are already being adapted to clinical trials, but existing approaches have drawbacks. In the next section, we discuss our vision of generative machine-learning models and digital twins in clinical trials.

The inventors realized that there are three obstacles to overcome when developing methods for implementing digital twins in a clinical trial context.
i. First, large multimodal data is needed, including genetic characterization, lab values, hospital admissions, diagnoses and drug prescriptions. Generative deep learning models thrive in large data settings, and can exploit the highly non-linear patterns found in multimodal data.
ii. Secondly, generative digital twins used currently are “black box” and interpreted only with post-hoc methods. By lacking a straightforward interpretation, it is challenging both for the public to trust the models and for developers to understand which components need improvement.
iii. Thirdly, the evaluation strategies of generated digital twin trajectories are rather limited, and there is especially a lack of relevant metrics, making it challenging to evaluate digital twin models. To address this, methods and public datasets for unbiased comparison should be developed jointly by machine learning and clinical trial experts.

Digital twin models raise a number of ethical and regulatory questions that need to be addressed. For example, how to ensure that clinicians and patients can trust digital twin predictions and the decisions made on their health. Furthermore, there is no specific regulation regarding the use of digital twins in clinical trials. For example, the Committee for Medicinal Products for Human Use (CHMP) from the EMA recently published a qualification opinion in which it qualified the use of digital twin predictions for supporting the statistical analysis of control arms, but this opinion assumes that the digital twins have been independently qualified.

However, no qualifications or requirements for digital twins in clinical trials themselves have been provided to date by the EMA or FDA. Digital twin researchers and regulators need to shape the requirements together to find a solution that is safe, technically feasible and impactful.

To conclude, current generative AI models have limitations, however, we are confident that these will be overcome in the near future. Generative AI will become a cornerstone technology enabling digital twins. It is our belief that the above outlined use cases encourage future developments by the scientific community, and digital twins will revolutionize clinical trials and drug development

### SUMMARY OF THE INVENTION

The present inventors propose to augment clinical trials with digital twins, which are virtual representations of patients that resemble the longitudinal characteristics of actual patients [4]. With the aid of digital twins, it becomes feasible to generate entire and realistic clinical patient trajectories [5]. Thus, there is a bidirectional connection between patients and their digital twins: information flows from the patient to their virtual digital twin representations to simulate its current and future states, as well as back from the digital twins to the patient to facilitate medical decision-making. Ideally, digital twins should be indistinguishable from real patients in their observed characteristics, such as their monitored clinical variables and disease prognoses.

Digital twins pave the way to significantly accelerate clinical trials. Data generated by digital twins could reduce long patient recruitment processes, e.g. basket trials of rare conditions which are often critically limited by the amount of recruited patients [6].

Another example are phase I & II clinical trials in oncology. In this case, digital twins can simulate comparator arms, and thereby enable efficacy assessment earlier. In essence, digital twins can increase statistical power through a higher number of simulated data, thus accelerating clinical decisions.

Digital twins can be realized in different forms, such as through mechanistic modelling [7] as well as using artificial intelligence [8]. Mechanistic approaches enable deep biological insights but require simulation parameters that are challenging to acquire in most clinical settings and are typically limited to only a subset of all available clinical variables.

Artificial intelligence algorithms can overcome these challenges, process all available clinical data and capture meaningful clinical associations [9]. The rapid development of computational resources, algorithmic advances and increased biomedical data availability is laying the foundation for generative artificial intelligence methods to revolutionize digital twins.

The present invention leverages the recent advances in computational power and the sophistication of generative artificial intelligence models in order to enable forecasting of various attributes of a subject in a clinical trial context. At a high level, the invention provides a computer-implemented method including receiving a medical history of a subject, which is used to initialize a generative model. Then, the model is run on the medical history data, and outputs values of desired attributes in a desired time frame. Computer-implemented methods according to the present invention thus have the potential to transform clinical trials and the process of drug discovery.

More specifically, a first aspect of the present invention provides a computer-implemented method of forecasting, predicting, or simulating values of selected subject-related attributes during a clinical trial, the computer-implemented method comprising: receiving input data comprising: a medical history of a subject, the medical history comprising values of plurality of subject-related attributes of a subject, the data comprising: one or more selected attributes of the subject and a time frame; applying a trained generative machine-learning model to the received input data, the trained generative machine-learning model configured to generate output data based on the input data, the output data comprising: respective values of the one or more specified attributes of the subject in the specified time frame.

In the context of the present application, the term “artificial intelligence” is used to refer to the multidisciplinary field that involves the development of agents capable of performing tasks that would ordinarily require human-level intelligence, such as speech recognition, decision-making, and experiential learning. The creation of such agents may involve the use of data and algorithms that allow computers to perceive, reason, and act in ways that emulate human cognition. A subfield of artificial intelligence is “machine-learning”, which is used to refer to the development of algorithms which are capable of learning. Generally, “machine-learning” focuses on the development of models that can analyse, cluster and interpret data, and make predictions based on provided input.

Throughout this application, we refer to a “model”, which term is used generally to refer to a mathematical representation of a system or a process characterized by parameters, for example to make predictions based on input data or determining overarching groupings of the input data. A “discriminative model” is a type of machine-learning model which may directly learn the relationship between input and output variables, without explicitly modelling the underlying probability distribution. Discriminative models are often used in tasks such as regression and classification. The present invention relies heavily on a “generative model”, which is generally used to refer to a type of machine-learning model which learns the underlying probability distribution of input variables, and can be used to generate new data similar to the training set. Generative models are often used in tasks such as image or text synthesis. The “architecture” of models may be referred to. “Architecture” refers to the structure of a machine-learning model, e.g. for a neural network this may include input and output layers, hidden layers of various sizes as well as further data transforms, activation functions, bias and computational operations.

In the context of machine-learning, a “neural network” or “artificial neural network” is a machine-learning model developed to mimic the structure and function of the human brain, consisting of interconnected nodes or “neurons” organized in layers. It may be trained on input data to learn patterns and relationships between the input and output data, and can be used for tasks such as classification, regression, and data generation. “Deep learning” machine-learning models are subsets of machine learning algorithms based on complex NN architectures, i.e. multiple hidden layers to model and solve complex problems arising from large and heterogeneous data. This approach has achieved remarkable breakthroughs in diverse domains, such as computer vision, natural language processing, and speech recognition.

When machine-learning models are trained, an approach referred to as a “training/test data split” may be employed. This is a technique in which a given dataset is divided into two parts, the training set and the test set, where the training set is used for building the model, whilst the test set is solely used to assess its generalizability to new, unseen data. Herein, “training” or “learning” refers to the iterative process of using input data to update the model's parameters by leveraging optimization algorithms to minimize a loss function. Once trained, the resulting model can be used for generating data, making predictions and, ultimately, patient relevant decisions.

According to the invention, the clinical input comprises a medical history of a subject, the medical history comprising a plurality of values of subject-related attributes of a subject. Because the computer-implemented method is applicable to clinical trials, it should be understood that the subject-related attributes are preferably attributes indicative of one or characteristics of a human being. Broadly speaking, these attributes may comprise clinical attributes, medical attributes, biological attributes, biomedical attributes, physiological attributes, genetic attributes, transcriptomic attributes, proteomic attributes, or the like. It is required that the plurality of values comprises values for at least one longitudinal attribute. A longitudinal attribute is an attribute whose value is measured a plurality of times, at different occasions, in order to track any changes in value of that attribute. The longitudinal attribute may be an attribute whose value changes with time. The plurality of subject-related attributes may comprise one or more longitudinal attributes, and thus the medical history may comprise one or more values of at least one longitudinal attribute. Preferably, the medical history may comprise a plurality of values of the one or more longitudinal attributes, each value corresponding to a measurement of the at least one longitudinal attribute at a respective (different) time. The subject-related attributes may comprise a plurality of longitudinal attributes, and the medical history may comprise, for each longitudinal attribute of the plurality of longitudinal attributes, a plurality of values of that longitudinal attribute, each value corresponding to a measurement of that longitudinal attribute at a respective (different) time point. In contrast, a static attribute is an attribute whose value is measured once, and is assumed not to change. An example of a static attribute is date of birth. A list of the attributes whose values may be specified is annexed to this patent application. The medical history may comprise at least 100 subject-related attributes, at least 200 subject-related attributes, at least 300 subject-related attributes, at least 400 subject-related attributes, at least 500 subject-related attributes, at least 600 subject-related attributes, at least 700 subject-related attributes, at least 800 subject-related attributes, at least 900 subject-related attributes, or at least 1000 subject-related attributes. For the longitudinal attributes, there may be at least 5 values per subject-related attribute, at least 10 values per subject-related attribute, at least 20 values per subject-related attribute, at least 50 values per subject-related attribute, at least 100 values per subject-related attribute, or at least 200 values per subject-related attribute.

Herein, the term “value” does not necessarily refer to a numerical value, but may also be used to refer any data specifying an attribute. For example, the value may be in the form of a date, a binary value (e.g. “YES” or “NO”, or Boolean operators such as “TRUE” or “FALSE”). The values may also take the form of descriptive words or statements, e.g. describing symptoms, side effects, or the like.

The trained generative machine-learning model may be a large language model (LLM). In the context of the present invention, a large language model is a computerized language model which may be embodied by an artificial neural network using an enormous number of parameters. A “language model” in this context is used to refer to a probability distribution over sequences of words. In implementations in which the large language model is embodied in an artificial neural network, the term “parameters” refers to the neurons in its layers, which may comprise a large number of weights between them. The large language model may comprise more than 10n parameters, where n is no less than 8, 9, 10, 11, 12, 13, 14, or 15.

There are various large language models which may be used in implementations of the present invention. Suitable large language models which may be used include:
T5—see Raffel et al. (2020) [23]
LongT5—see Guo et al. (2021°) [24]
MPT—see [25]
Pegasus-X—see Phang et al. (2022) [26]
Longformer—see Beltagy et al. (2020) [27]
GPT-1—see Radford et al. [28]
GPT-2—see Radford et al. (2019) [29]
GPT-3—see Brown et al. (2020) [30]
GPT-3.5—see [31]
GPT-4—see [32]
Hyena—see Poli et al. (2023) [33]
LLAMA—see Touvron et al. (2023) [34]
falcon-see [35]

Commercially available LLMs are typically trained on a vast corpus of data, obtained from the Internet. While this training data may include the kind of medical information which is useful for forecasting the values of various subject-related attributes in a clinical trial context, it is possible to improve the performance of the LLM (or other generative model) further by training it in a supervised manner using training data which is more closely related to the context in which the LLM is to be used, according to various implementations of the present invention. The training data may comprise the Flatiron data set.

Accordingly, the generative machine-learning model of the present invention may have been trained using a computer-implemented method comprising: receiving a partially trained generative machine-learning model; and training the partially trained generative machine-learning model in a supervised manner using training data comprising a plurality of medical histories, each medical history comprising: for a given subject, data indicative of the values of a plurality of subject-related attributes. Herein, “partially trained” is to be understood to mean that the generative machine-learning model has been trained, for example, only on a large corpus of general data, rather than training data which is specific to its application in the context of a clinical trial. The training data may comprise at least 100 medical histories, at least 1,000 medical histories, at least 10,000 medical histories, at least 100,000 medical histories, or at least 1,000,000 medical histories.

Given that implementations of the computer-implemented method of the first aspect of the invention are intended for forecasting the values of subject-related attributes, it is advantageous for the medical histories which form part of the training data to comprise values of longitudinal attributes. Accordingly, the plurality of subject-related attributes may comprise one or more longitudinal attributes, and thus the training data may comprise one or more values of at least one longitudinal attribute. Preferably, the training data may comprise a plurality of values of the one or more longitudinal attributes, each value corresponding to a measurement of the longitudinal attribute at a respective (different) time. The subject-related attributes may comprise a plurality of longitudinal attributes, and the training data may this comprise, for each longitudinal attribute of the plurality of longitudinal attributes, a plurality of values of that longitudinal attribute, each value corresponding to a measurement of that longitudinal attribute at a respective (different) time.

Large language models that are trained on text documents are best equipped to handle input data and training data which are expressed in natural language, rather than, for example, tabular data. It is therefore advantageous to use data in a particular form, or syntax, for the supervised training of the partially trained generative machine-learning model, particularly in those cases where the partially trained generative machine-learning model is a large language model. Accordingly, training the generative machine-learning model may further comprise: receiving raw training data. The raw training may be in the form of tabular data. Then, training the generative machine-learning model may further comprise: converting the raw training data to training data having a predetermined syntax or structure that is appropriate for input into the generative machine-learning model.

We now discuss various features of one such predetermined syntax.

Firstly, the converted training data may be in a Javascript Object Notation (JSON) format. JSON is an open standard file format and data interchange that uses human-readable text to store and transmit data objects consisting of attribute-value pairs and arrays. The JSON format is particularly useful for the present invention because it is well-equipped to handle the attribute-value pairs which are inherent to the effectiveness of the invention.

Within the converted training data, the JSON may comprise a first portion and a second portion, wherein the first portion of the JSON comprises data defining the values of the longitudinal attributes and the second portion of the JSON comprises data defining the values of the static attributes. Within the first and second portions, the attributes are preferably assigned identifiers which are descriptive and unique. By using descriptive identifiers, the generative machine-learning model (which has been partially trained on a vast corpus of general data) will better be able to draw associations between features of the converted training data and features from the vast corpus of general data used to generate the partially trained model. By using unique labels, the risk of confusion between different subject-related attributes is minimized or eliminated.

Medical histories generally comprise various measurements taken on different days. The set of measurements taken on one day may be different from the measurements taken on another day. However, generally each set of measurements comprises a date on which the measurements were taken. In the predetermined syntax, it is preferable that relative, rather than absolute, dates are employed. Specifically, rather than specifying that a given set of measurements were taken on e.g. 1 Jan. 2020, within the converted training data, it would be specified that the given set of measurements were taken on Day 0 (or, equivalently Day 1). Then, the dates of all other measurements would be expressed relative to the earlier date. For example, another set of measurements taken on 1 Feb. 2020 may be labelled Day 31 or “31 days later”. Alternatively, rather than being expressed relative to the earliest date, the dates may be expressed relative to the previous date for which there is data in the medical history.

The use of relative dates and times in this manner minimizes overfitting of the generative machine-learning model during by supervised training (equivalently referred to as supervised learning), by removing the risk that, during training, the model associates various features with the absolute dates, rather than the progression of time.

Converting the raw training data into converted training data having the predetermined syntax may comprise applying a conversion algorithm to the raw training data, which may be in tabular form. Specifically, the conversion algorithm may be configured to execute the following steps on the raw training data (either in the order set out below, or in any other order):
The conversion algorithm may comprise a step of identifying or extracting data defining the values of static attributes (referred to as “static data”, for brevity) and data defining the values of longitudinal attributes (referred to as “longitudinal data”, for brevity).
The conversion algorithm may comprise a step of opening, generating, and/or initializing a JSON object.
Then, for the longitudinal data, the conversion algorithm may comprise: identifying a first subset of the longitudinal data which corresponds to measurements obtained on a first date, the first subset of longitudinal data comprising a first absolute value identifying the first date. The conversion algorithm may comprise converting the first absolute value to a first relative value indicating that it is the earliest date, for example “Day 0” or “0 Days Later”. Having generated this value, the algorithm may proceed to generate a value of a JSON dictionary for the first relative value. Then, for every measurement obtained on the first date, the conversion algorithm may comprise: converting the measurement identifier into a descriptive and unique identifier. This may comprise executing a lookup for the raw measurement name in a lookup table to find the corresponding predetermined descriptive and unique name. The conversion algorithm may then comprise generating and storing associations between the measurement identifiers and the respective values of the subject-related attributes (corresponding to those measurements) in the JSON dictionary entry corresponding to the first relative value. If there is no data for a given measurement identifier, this may be skipped. Thus a JSON dictionary entry corresponding to the first relative value is created.
The conversion algorithm may further comprise: identifying a second subset of the longitudinal data which corresponds to measurements obtained on a second date (later than the first date), the second subset of longitudinal data comprising a second absolute value identifying the second date; and converting the second absolute value to a second relative value based on a difference between the second absolute value and the first absolute value. Having generated the second relative value, the algorithm may proceed to generate a value of a JSON dictionary for the second relative value. Then, for every measurement obtained on the second date, the conversion algorithm may comprise: converting the measurement identifier into a descriptive and unique identifier. This may comprise executing a lookup for the raw measurement name in a lookup table to find the corresponding predetermined descriptive and unique name, as before. The conversion algorithm may then comprise generating and storing associations between the measurement identifiers and the respective values of the subject-related attributes (corresponding to those measurements) in the JSON dictionary entry corresponding to the second relative value. If there is no data for a given measurement identifier, this may be skipped. Thus a JSON dictionary entry corresponding to the second relative value is created.
The above steps may be repeated as necessary for additional dates, i.e. converting an n-th absolute date into an n-th relative value (which may be relative to the first relative value, or relative to the date corresponding to the (n−1)th relative value), and for each measurement obtained on the n-th date, converting the measurement identifier into a descriptive and unique identifier, which may comprise executing a lookup for the raw measurement name in a lookup table to find the corresponding predetermined descriptive and unique name. The conversion algorithm may then comprise generating and storing associations between the measurement identifiers and the respective values of the subject-related attributes (corresponding to those measurements) in the JSON dictionary entry corresponding to the n-th relative value. If there is no data for a given measurement identifier, this may be skipped. Thus a JSON dictionary entry corresponding to the n-th relative value is created.
At this juncture, the JSON object comprises, in a first portion, a dictionary corresponding to the longitudinal data, the dictionary listing each relative value, each dictionary entry comprising data defining the values of a plurality of longitudinal attributes for various dates, the dates expressed in relative terms.
The conversion algorithm may also comprise: generating a JSON dictionary corresponding to the static data in a second portion of same JSON object. This may comprise, for each static attribute, converting the measurement identifier into a descriptive and unique identifier. This, in turn, may comprise executing a lookup for the raw measurement name in a lookup table to find the corresponding predetermined descriptive and unique name, as before. The conversion algorithm may then comprise generating and storing associations between the measurement identifiers and the respective values of the statis attributes (corresponding to those measurements) in the JSON dictionary entry corresponding to the static data. If there is no data for a given measurement identifier, this may be skipped. Thus a JSON dictionary entry corresponding to the static data is created.

The output of the conversion algorithm is thus a JSON object containing the data from the raw training data, arranged in a specific manner which is particularly applicable to the training of generative machine-learning models, in particular large language models.

Alternatively, rather than using a conversion algorithm which executes a series of steps as outlined above, the conversion algorithm itself may be in the form of a trained machine-learning model which is trained to convert raw data (in any form) into converted training data in the predetermined syntax. Specifically, the trained machine-learning model may have been trained using training data which is generated using the conversion algorithm outlined above. More generally, the training data may comprise a plurality of records, each record comprising raw data as an input, and output data comprising a representation of the raw data in the desired predetermined syntax. The trained machine-learning model may be in the form of an artificial neural network model, such as a general recurrent neural network (e.g. LSTMs, GRUs), convolutional neural network, or neural ordinary differential equation (ODE).

Alternatively, the trained machine-learning model may itself be in the form of a large language model, or a transformer.

There are significant technical advantages associated with training the generative machine-learning model using data which has been converted into the predetermined syntax as outlined above. Generally, training data, such as the tabular data which may form the raw training data may originate from several sources. Each source may use, for example, different identifiers for different measurements, and may include different measurements altogether. As a result, the raw training data may be inconsistent and messy. Large language models are generally trained on such a vast corpus of data that they are essentially able to handle any inconsistencies like this. However, they are not generally equipped to receive tabular data as their input. So, by converting the training data into a consistent form having an appropriate predetermined syntax, it is possible to leverage the capabilities of large language models to handle otherwise messy, inconsistent training data, and to deliver improved results.

We have discussed the training of the generative machine-learning model in detail. We now discuss the application of the generative machine-learning model in more detail.

The input data comprises the medical history of the subject, as well as data specifying a requested output, specifically one or more subject-related attributes whose value a user wishes to forecast, and a time frame over which to forecast the values of the one or more subject-related attributes. It is preferable that the input data takes the same form as the training data. We have discussed already in detail a preferable form for the training data in order to enable execution of the computer-implemented method of the present invention to leverage the capabilities of large language models and generative machine-learning models in general. Accordingly, before application of the generative machine-learning model, the computer-implemented method may further comprise converting the received input data into converted input data having the predetermined syntax which is appropriate for input into the generative machine-learning model. For completeness, we repeat the details of the conversion and the predetermined syntax here.

Firstly, the converted input data may be in a JavaScript Object Notation (JSON) format.

Within the converted input data, the JSON may comprise a first portion, a second portion, and a third portion, wherein the first portion of the JSON comprises data defining the values of the longitudinal attributes, the second portion of the JSON comprises data defining the values of the static attributes, and the third portion comprises data defining the desired output. Within the first, second, and third portions, the subject-related attributes are preferably assigned identifiers which are descriptive and unique. The training data may also take this form, in order to ensure that it the generative machine-learning model is configured to output data in the correct format. For example, even if the training data includes information about the desired output subject-related attributes, the model will preferably be trained by structuring the training data in a manner where these are expressed in the form of “desired variables”, to ensure that the generative machine-learning model is able to learn that these are output variables, and to structure the output correctly.

Specifically, the third portion of the JSON object may comprise the data defining the subject-related attributes whose values are to be forecast, and a time frame. In the predetermined syntax, as for the training data, it is preferable that relative, rather than absolute, dates are employed.

Converting the input data into converted input data having the predetermined syntax may comprise applying a conversion algorithm to the input data, which may be in tabular form. Specifically, the conversion algorithm may be configured to execute the following steps on the input data (either in the order set out below, or in any other order):
The conversion algorithm may comprise a step of, within the medical history, identifying or extracting data defining the values of static attributes (referred to as “static data”, for brevity) and data defining the values of longitudinal attributes (referred to as “longitudinal data”, for brevity).
The conversion algorithm may comprise a step of opening, generating and/or initializing a JSON object.
Then, for the longitudinal data in the medical history, the conversion algorithm may comprise: identifying a first subset of the longitudinal data which corresponds to measurements obtained on a first date, the first subset of longitudinal data comprising a first absolute value identifying the first date. The conversion algorithm may comprise converting the first absolute value to a first relative value indicating that it is the earliest date, for example “Day 0” or “0 Days Later”. Having generated this value, the algorithm may proceed to generate a value of a JSON dictionary for the first relative value. Then, for every measurement obtained on the first date, the conversion algorithm may comprise: converting the measurement identifier into a descriptive and unique identifier. This may comprise executing a lookup for the raw measurement name in a lookup table to find the corresponding predetermined descriptive and unique name. The conversion algorithm may then comprise generating and storing associations between the measurement identifiers and the respective values of the subject-related attributes (corresponding to those measurements) in the JSON dictionary entry corresponding to the first relative value. If there is no data for a given measurement identifier, this may be skipped. Thus a JSON dictionary entry corresponding to the first relative value is created.
The conversion algorithm may further comprise: identifying a second subset of the longitudinal data which corresponds to measurements obtained on a second date (later than the first date), the second subset of longitudinal data comprising a second absolute value identifying the second date; and converting the second absolute value to a second relative value based on a difference between the second absolute value and the first absolute value. Having generated the second relative value, the algorithm may proceed to generate a value of a JSON dictionary for the second relative value. Then, for every measurement obtained on the second date, the conversion algorithm may comprise: converting the measurement identifier into a descriptive and unique identifier. This may comprise executing a lookup for the raw measurement name in a lookup table to find the corresponding predetermined descriptive and unique name, as before. The conversion algorithm may then comprise generating and storing associations between the measurement identifiers and the respective values of the subject-related attributes (corresponding to those measurements) in the JSON dictionary entry corresponding to the second relative value. If there is no data for a given measurement identifier, this may be skipped. Thus a JSON dictionary entry corresponding to the second relative value is created.
The above steps may be repeated as necessary for additional dates in the medical history, i.e. converting an n-th absolute date into an n-th relative value (which may be relative to the first relative value, or relative to the date corresponding to the (n−1)th relative value), and for each measurement obtained on the n-th date, converting the measurement identifier into a descriptive and unique identifier, which may comprise executing a lookup for the raw measurement name in a lookup table to find the corresponding predetermined descriptive and unique name. The conversion algorithm may then comprise generating and storing associations between the measurement identifiers and the respective values of the subject-related attributes (corresponding to those measurements) in the JSON dictionary entry corresponding to the n-th relative value. If there is no data for a given measurement identifier, this may be skipped. Thus a JSON dictionary entry corresponding to the n-th relative value is created.
At this juncture, the JSON object comprises, in a first portion, a dictionary corresponding to the longitudinal data forming part of the medical history, the dictionary listing each relative value, each dictionary entry comprising data defining the values of a plurality of longitudinal attributes for various dates, the dates expressed in relative terms.
The conversion algorithm may also comprise: generating a JSON dictionary corresponding to the static data which forms part of the medical history in a second portion of same JSON object. This may comprise, for each static attribute, converting the measurement identifier into a descriptive and unique identifier. This, in turn, may comprise executing a lookup for the raw measurement name in a lookup table to find the corresponding predetermined descriptive and unique name, as before. The conversion algorithm may then comprise generating and storing associations between the measurement identifiers and the respective values of the static attributes (corresponding to those measurements) in the JSON dictionary entry corresponding to the static data. If there is no data for a given measurement identifier, this may be skipped. Thus a JSON dictionary entry corresponding to the static data is created.
At this point, the data in the medical history has been converted into an appropriate form in the JSON object. In addition, the input data specifies one or more subject-related attributes whose value is to be forecast and a time frame. Accordingly, the conversion algorithm may further comprise generating, in the third portion of the JSON object, an additional dictionary entry comprising data identifying the one or more subject-related attributes whose values are to be predicted. And, the conversion algorithm may further comprise generating, in the third portion of the JSON object, a further dictionary entry comprising data defining the time frame within which the values of the specified subject-related attributes should be forecast. As discussed, this is preferably in the form of a relative value, rather than an absolute date.
The output of the conversion algorithm is thus a JSON object containing the data from the medical history which forms part of the input data, arranged in a specific manner which is particularly applicable to the application of generative machine-learning models, in particular large language models, along with data in a similar format which indicates the desired output of the application of the generative machine-learning model.

Alternatively, rather than using a conversion algorithm which executes a series of steps as outlined above, the conversion algorithm itself may be in the form of a trained machine-learning model which is trained to convert the input data (in any form) into converted input data in the predetermined syntax. Specifically, the trained machine-learning model may have been trained using training data which is generated using the conversion algorithm outlined above. More generally, the training data may comprise a plurality of records, each record comprising raw input data as an input, and output data comprising a representation of the raw input data in the desired predetermined syntax. The trained machine-learning model may be in the form of an artificial neural network model, such as a general recurrent neural network (e.g. LSTMs, GRUs), convolutional neural network, neural ordinary differential equation (ODE). Alternatively, the trained machine-learning model may itself be in the form of a large language model, or a transformer.

Computer-implemented methods according to the first aspect of the invention are for use in the context of clinical trials. As such, it may be desirable to make predictions based on an indication of a therapeutic intervention. Herein, the term “therapeutic intervention” is used broadly to refer, for example, to pharmaceutical treatments, as well as other interventions such as transplants and other surgeries, and behavioural interventions. For example, a clinician may wish to use the computer-implemented method of the invention to forecast a patient's response to a particular therapeutic intervention, such as a standard-of-care intervention. In this way, the forecast can act, effectively, as a control in a clinical trial. By executing a digital control in this manner, great savings can be made in terms of resources, and time. This also avoids the need for some candidates on a clinical trial not to be given any treatment at all.

Accordingly, the data specifying a requested output may further comprise data identifying a therapeutic intervention. In this way, the generative machine-learning model may be configured to generate an output which is indicative of the values of the one or more specified subject-related attributes if the subject had been taking or treated using the identified therapeutic intervention. The data identifying the therapeutic intervention may comprise, for example, the type of therapeutic intervention, e.g. an identifier of a drug or other pharmaceutical treatment and a dosage or more specifically a dosage regime, where necessary. The data identifying the therapeutic intervention may form part of the third portion of the JSON object. The therapeutic intervention need not be related to a single intervention, and thus may also be a combination therapeutic intervention, e.g. in the form of more than one drug, or a drug and other treatment. In order reliably to forecast the effect of a given therapeutic intervention, the generative machine-learning model should be trained on data relating to subjects who have been treated using that, or similar, therapeutic intervention. Specifically, the training data may comprise a plurality of medical histories relating to subjects who have been treated using the therapeutic intervention, the medical histories comprising data indicating that the subjects have been treated using the therapeutic intervention. Where necessary, the data indicating that the subjects have been treated using the therapeutic intervention may comprise an indication of the therapeutic intervention and a dosage regime. It is not necessary that all of the medical histories making up the training data relate to subjects who have been treated using the therapeutic intervention.

The therapeutic intervention may comprise a treatment for cancer. The therapeutic intervention may comprise a treatment for inflammatory bowel disease. The therapeutic intervention may comprise a treatment for a neurodegenerative condition such as Parkinson's disease, multiple sclerosis, or Alzheimer's disease. The therapeutic intervention may comprise a treatment for nephropathy.

Using computer-implemented methods of the present invention, it is possible to make predictions about the values of various subject-related attributes in all manner of time frames. Specifically, the values of the one or more longitudinal attributes may comprise data corresponding to: a value of the one or more longitudinal attributes at an earliest time; and a value of the one or more longitudinal attributes at a latest time; and the time frame corresponds to: a time before the earliest time; a time between the earliest time and the latest time; or a time later than the latest time. In this way, computer-implemented methods according to the present invention may be used to predict values of the desired subject-related attribute at any point in time, e.g. before the medical history, after the medical history, or at a point during the medical history for which no measurements are available, or such data is missing.

The output data comprises values of the one or more specified subject-related attributes of the subject in the specified time frame. By adding additional steps to the computer-implemented method, it is possible to obtain a predicted trajectory for the one or more specified subject-related attributes. Below, we explain the process for one subject-related attribute, but it will be readily appreciated that the same method may be applied for some, any or all of the specified subject-related attributes. More specifically, a predicted trajectory may be obtained by recursively applying the generative machine-learning model, i.e. by adding the output value of the model to the input data to generate modified input data and applying the generative machine-learning model to the modified input data. This recursive process may be repeated for a predetermined number of iterations, or until an end condition is met.

More specifically, the computer-implemented method may further comprise, after the output data has been generated: generating modified input data by combining the input data with the output data; and applying the trained generative machine-learning model to the modified input data to generate updated output data. The computer-implemented method may then further comprise determining whether an end condition is met. If it is determined that the end condition has not been met, the computer-implemented method may further comprise repeating the steps of generating modified input data, applying the model to the modified input data and determining whether the end condition is met. This may repeat until it is determined that the end condition is met.

If it is determined that the end condition has been met, the computer-implemented method may then comprise outputting the data. Outputting the data may comprise outputting the updated output data generated in the most recent step, or alternatively, may comprise outputting data comprising the output data and updated output data from each step, for example in the form of a graph, or trajectory.

This process may be repeated until output data corresponding to the specified time frame has been output, or until the process has been repeated a predetermined number of times (i.e. these may be the end conditions in question).

From the above, it will be appreciated that the present invention may be employed in a clinical trial context or a drug discovery context by generating results for a control arm of the clinical trial. The safety and/or efficacy of the therapeutic intervention being investigated in the clinical trial may then be determined by comparing the results of the clinical trial with the digitally generated control results. An output of such a comparison may then be used to inform future decisions during the drug discovery, development, design, or manufacture process, as well as a process for determining dosage regimes. Accordingly, a second aspect of the present invention provides a computer-implemented invention of determining an efficacy and/or safety of a trial therapeutic intervention in a clinical trial, the computer-implemented method comprising: receiving electronic data comprising the results of a clinical trial relating to a trial therapeutic intervention; receiving control data, the control data generated by executing the computer-implemented method of the first aspect of the invention, the control data comprising the generated output data; determining an efficacy and/or safety of the trial therapeutic intervention based on a comparison of the electronic data comprising the results of the clinical trial with the control data comprising the generated clinical output data. In some cases, a categorical variable indicative of disease response may be used. The variable may take values such as “stable disease”, “partial response”, “progressive disease” etc. In order to determine an efficacy, each class may have an associated weight, and the efficacy is determined based on the calculated weights. Alternatively, an efficacy may be determined based on a number of state switches.

In these cases, the control data may be generated for a control therapeutic intervention or for no therapeutic intervention. The control therapeutic intervention may be a standard-of-care therapeutic intervention or a placebo. The method may be executed for each subject in the clinical trial in order to enable a “like for like” comparison. Equivalently, the results of the clinical trial may comprise values of a plurality of subject-related attributes at a plurality of points in time. In order to enable a valid comparison, the control data preferably comprises values of at least one subject-related attribute of the plurality of subject-related attributes (comprised in the clinical trial results) and more preferably values of the same plurality of subject-related attributes. Preferably, the control data comprises values of the plurality of subject-related attributes corresponding to the same time frame, if not exactly the same time points.

Based on the comparison between the control data and the results of the clinical trial, the computer-implemented method of the second aspect of the invention may further comprise determining a value of an efficacy and/or safety metric indicative of the efficacy and/or safety of the trial therapeutic intervention. The computer-implemented method may further comprise selecting the trial therapeutic intervention for further investigation based on the value of the efficacy and/or safety metric. The computer-implemented method of the second aspect of the invention may be executed in respect of a plurality of trial therapeutic interventions, and a respective efficacy and/or safety metric may be determined for each trial therapeutic intervention of the plurality of trial therapeutic interventions. Then, the computer-implemented method may further comprise selecting a trial therapeutic intervention of the plurality of trial therapeutic interventions for further investigation based on the determined efficacy and/or safety metrics. Herein, the different trial therapeutic interventions may comprise different therapies, or may comprise different dosages of the same therapy.

The two aspects of the invention outlined above are directed towards computer-implemented methods. Additional aspects of the invention include:
A forecasting system comprising a processor, wherein the processor is configured to execute the computer-implemented method of the first aspect of the invention and/or the computer-implemented method of the second aspect of the invention.
A computer program (or computer program product) comprising instructions which, when the program is executed by a computer or a processor thereof, cause the computer to carry out the computer-implemented method of the first aspect of the invention and/or the computer-implemented method of the second aspect of the invention.
A computer-readable storage medium comprising instructions which, when executed by a computer, cause the computer to carry out the steps of the computer-implemented method of the first aspect of the invention and/or the computer-implemented method of the second aspect of the invention.

The optional features set out in this application in respect of the first aspect of the invention or the second aspect of the invention are equally applicable to all other aspects of the invention.

The invention includes the combination of the aspects and preferred features described except where such a combination is clearly impermissible or expressly avoided.

### BRIEF DESCRIPTION OF THE DRAWINGS

Embodiments of the present invention will now be described with reference to the accompanying drawings, in which:

FIG. 1 shows an example of a system 10 which may be used to execute computer-implemented methods of the present invention.

FIG. 2A is a flowchart illustrating a high-level training process for a generative machine-leaning model.

FIG. 2B is a flowchart illustrating an example of a supervised learning process.

FIG. 3 is an example of a JSON object comprising training data.

FIG. 4 is a flowchart illustrating a high-level model application process according to the present invention.

FIG. 5 is an example of a JSON object comprising a medical history of a subject and data specifying a requested output of a generative machine-learning model.

FIG. 6 is an example of a JSON object comprising an output of a generative machine-learning model.

FIG. 7 is a flowchart illustrating a recursive/iterative method which may be used to output a series of output points.

FIG. 8 shows some use cases of computer-implemented methods of the present invention.

FIG. 9, panels a-j shows how generative digital twins (DTs) can be realized by various deep learning (DL) architectures. (panel a) Input data consisting of patient history. (panel b) Uniform Manifold Approximation and Projection (UMAP) applied to the last layer of a discriminative model predicting the probability of toxicity. (panel c) Dimensionality reduction method UMAP applied to the last layer of a generative DT model at time t+1 of the predicted future patient trajectory. (panel d) The flow of information between DTs and real patients is bidirectional, as DTs are virtual representations of patients that can help improve patient treatment. Simplified visualization of existing generative DT architectures: (panel e) Conditional restricted Boltzmann machine (CRBM) and (panel f) variational autoencoder (VAE). Potential generative DT architectures are (panel g) generative adversarial networks (GAN), (panel h) stable diffusion, (panel i) neural ordinary differential equations (neural ODE) and (panel j) transformers.

### DETAILED DESCRIPTION OF THE DRAWINGS

Aspects and embodiments of the present invention will now be discussed with reference to the accompanying figures. Further aspects and embodiments will be apparent to those skilled in the art. All documents mentioned in this text are incorporated herein by reference.

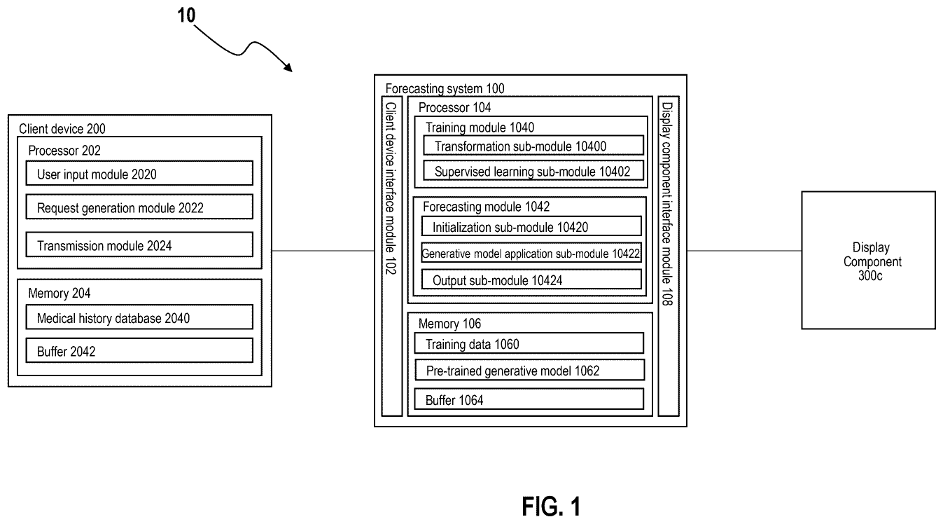

FIG. 1 shows an example of a system 10 which may be used to execute various computer-implemented methods according to the present invention. The system 10 includes a forecasting system 100, a client device 200 and a display component 300. These may all be separate components, in which case they may be connected via some kind of network (not shown), via a wireless connection, a wired connection, or a mixture of the three. When the forecasting system 100, client device 200, and display component 300 are connected via a network, the network may be a wireless network such as a wireless Internet connection, a Wi-Fi network, a cellular network or any other suitable or equivalent network. Alternatively, the network may be a wired network such as a LAN, a wired Internet connection, or a WLAN. The skilled person readily appreciates that other kinds of network connection are possible.

We now discuss the forecasting system 100 in more detail. It should be noted that the forecasting system 100 may equivalently be referred to as a prediction system, or a simulation system. It will be noted that the forecasting system 100 comprises several “modules” and “sub-modules”. The forecasting system 100 as a whole may be implemented either in the form of bespoke hardware, or more likely the forecasting system 100 may be implemented in software, for example in the form of computer-readable code comprising instructions which, when executed, cause a computer to execute the various functions described herein. Similarly, the modules (described in more detail later) may also be implemented in the form of hardware modules within the processor 104, but may also be implemented in the form of software modules. The software modules may be represented, for example, by computer code comprising instructions which, when executed, cause the computer to execute the respective function associated with that module. In this sense, the modules may be interpreted as “functional modules”, which may be implemented in any computer-based manner, such that they are able to execute the function with which they are associated. In an abundance of caution, we note that the whole of the forecasting module 100 may be implemented on a general-purpose computer such as a desktop computer, a laptop computer, a smartphone, a tablet, or the like.

The forecasting module 100 comprises client device interface module 102, processor 104, memory 106, and display component interface module 108. As the name suggests, the purposes of the client device interface module 102 and the display component interface module 108 are to interface with the client device 200, and the display component 300, respectively. The client device interface module 102 and the display component interface module 108 may be implemented in any suitable form, be it a software module, a physical interface (such as a USB connection, or similar), or a network component configured to receive data-containing signals from the client device 200, or the display component 300. The client device interface module 102 and the display component interface module 108 may be the same component.

The processor 104 comprises a plurality of functional modules. Specifically, the processor 104 comprises a training module 1040 and a forecasting module 1042. In the implementation shown in FIG. 1, the training module 1040 comprises a transformation sub-module 10400 and a supervised learning sub-module 10402, and the forecasting module 1042 comprises an initialization sub-module 10420, a generative model application sub-module 10422, and an output sub-module 10424.

The memory 106 of the forecasting system 100 stores training data 1060, a pre-trained generative model 1062 and a buffer 1064. The buffer 1064 takes its normal role, i.e. temporarily storing or caching received data so that it may be retrieved for processing, by the processor 104, more rapidly.

The specific implementation of the forecasting system 100 (including the processor 104 and the memory 106) shown in FIG. 1 is an illustrative example only, and it will be appreciated from the preceding disclosure that the processor 104 of the forecasting system 100 need not include some or all of the functional modules shown, or alternatively may including any sub-combination of functional modules. All sub-combinations are envisaged.

The client device 200 comprises a processor 202, which itself comprises a user input module 2020, a request generation module 2022, and a transmission system 2024. The client device 200 further comprises a memory 204, which comprises a medical history database 2040 and a buffer 2042.

We now discuss various computer-implemented methods which may be executed by the system 10 shown in FIG. 1. Of course, methods or computer-implemented methods of the present invention may be executed by hardware or software arranged differently from the forecasting system 100 of FIG. 1. In the following, however, we will refer to the forecasting system 100, but the invention is not limited to such an arrangement.

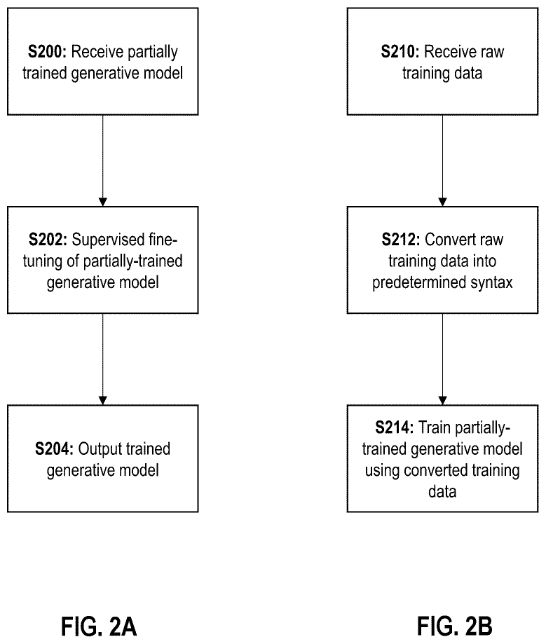

At the heart of the present invention is the application of a generative model to input data, in order to receive a clinically meaningful output. In order to ensure that the generative model performs effectively, it must first be trained using the training module 1042 of the processor 104 of the forecasting system 100. FIGS. 2A and 2B are flowcharts illustrating exemplary training processes. FIG. 2A is a high-level process for training a generative model, and FIG. 2B shows in more detail a series of steps which may be used in the supervised fine-tuning step of FIG. 2A.

In FIG. 2A, in a first step S200, a partially trained generative model is received at e.g. the training module 1040 of the processor 104 of the forecasting system 100. Typically, the partially trained generative model is a large language model which has been trained on the general corpus of data which can be mined from public sources such as the internet. Herein, “partially trained” is used to refer to a generative model which has not been trained in a supervised manner using data which is specific to the application of the model. In the present case, the data which is specific to the application of the model refers to the medical, clinical, biological, molecular, genetic, genomic, transcriptomic, proteomic data or the like. The partially trained generative model may be a publicly available model, or may be a bespoke model designed with this purpose in mind.

In step S202, the partially trained generative model is fine-tuned in a supervised manner. Herein, we refer to “supervised” training, or equivalently “supervised learning” as the process in which the partially trained generative model is trained using the training data 1060 which is relevant for the intended use of the generative model. As discussed in the previous paragraph, the partially trained model is trained using a general corpus of data mined, usually, from the Internet, but in step S202, the relevant medical, clinical, biological, molecular, genetic, genomic, transcriptomic, proteomic data or the like, is used. Specifically, in step S202, the supervised learning sub-module 10402 of the training module 1040 of the processor 104 of the forecasting system 100 retrieves the training data 1060 from the memory 106 of the forecasting system 100, and trains the generative model using it.

FIG. 2B shows a flowchart which illustrates the manner in which the fine-tuning process of step S202 of FIG. 2A may take place, in an implementation in which the generative model is in the form of a large language model, LLM. LLMs are generative models which specialize in the handling of language inputs, and accordingly, they are most efficiently trained using sentence-like inputs, rather than e.g. numerical arrays. However, the majority of the kind of data which is useful for training a generative model to forecast or predict future events in clinical trials is tabular data, rather than sentence-like data. Accordingly, before the raw training data can be used to train the generative model, the method of FIG. 2B includes a step of converting raw training data to have a predetermined syntax.

In step S210, the raw training data is received at the transformation sub-module 10400 of the processor 1040 of the processor 104 of the forecasting system 100. Then in step S212, the transformation sub-module 10400 applies an algorithm to the raw training data to convert into training data having a predetermined syntax which is appropriate for the training of the generative model. In the case of a large language model, raw tabular training data may be converted to sentence-like data using an algorithm having steps as set out below:
1. Extract data relating to static (e.g. date of birth) attributes and data relating to longitudinal attributes (e.g. heart rate measurement on 05.05.2023) data.
2. For longitudinal data, execute the following steps for each day where a measurement has been taken:
   i. Convert absolute date to the relative data from the previous measurement (e.g. if previous measurement was on 01.05.2023 and the current measurement is on 05.05.2023→convert to “4 days later”). If it is the first measurement, use “0 days later”.
   ii. For every measurement, convert the measurement name into a unique, descriptive name. This may be performed using a lookup table, which may be manually generated.
3. Append data relating to static attributes (alternatively referred to as “baseline data”), converting the measurement names to a unique, descriptive name, in the same manner as above.

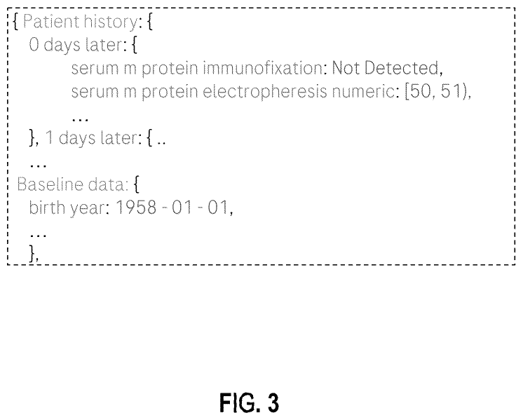

FIG. 3 shows an example of training data which has been transformed using the above algorithm. In the example of FIG. 3, the raw training data has been transformed into a JSON file. JSON is an open standard file format and data interchange that uses human-readable text to store and transmit data objects consisting of attribute-value pairs and arrays. In FIG. 3, the JSON object is separated into patient history, which contains data describing longitudinal clinical variables, and baseline data, which contains data describing static variables. Within the patient history, each set of clinical data is divided into a different time frame, including data from “0 days later” (i.e. the first measurements) and data from “1 days later”. Within each time frame, the values of various subject-related attributes are specified, including serum m protein immunofixation and serum m protein electrophoresis numeric. Within the baseline data, there are various attributes including birth year. As discussed elsewhere in this patent application, presentation of data in this manner allows a large language model to be trained using raw training data which is in tabular (or other form). It should be stressed that this is just one form that the training data can take, and other forms are equally applicable.

The training data may further comprise data specifying the subject-related attributes whose values are to be predicted, forecast or simulated. The training data may further comprise the time frame over which the prediction, forecast or simulation is to cover. Furthermore, by including the desired output data in the training data in this manner, the generative machine-learning model is able to learn how actually to deal with the inputs. In this manner, the training data may even more closely resemble the input data, and may take the form shown in FIG. 6, for example (described later with reference to conversion of the input data).

Returning to FIG. 2B, in step S214 the partially trained model is trained using the transformed training data using the supervised learning sub-module 10402 of the training module 1040 of the processor 104 of the forecasting system 100. Steps S210 to 214 of FIG. 2B are an example of a process which may be used to execute step S202 of FIG. 2A. After this has been completed, the computer-implemented method proceeds to step S204 in which the trained generative model 1062 is output.

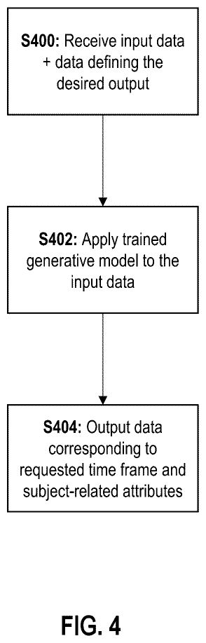

FIG. 4 illustrates an example of a process by which the forecasting system 100 may be used to apply the trained generative model 1060 to forecast a value of a requested subject-related attribute. In step S400 of FIG. 1, input data is received at the forecasting system 100 from the client device 200 via the client device interface module 102 of the forecasting system 100. Herein, the “input data” refers to data which may comprise the patient's medical history, which may include various forms of data, including both static data and longitudinal data. More specifically, in this step, the client device 200, more specifically the user input module 2020 of the processor 202 of the client device 200 may receive a user input. In one implementation, the user input may comprise a subject identifier, or an identifier of a medical history of a subject of interest. In response, the processor 202 may retrieve a medical history from the medical history database 2040. The request generation module 2022 of the processor 202 of the client device 200 is then configured to generate the request to be sent to the forecasting system 100. While the request is being generated by the request generation module 2022, it may be stored in the buffer 2024. After the request is generated, it may be transmitted by the transmission module 2024, whereupon it is received at the forecasting system 100 via the client device interface module 102.

Like when training the generative model 1060, as illustrated in FIGS. 2A and 2B, it is also advantageous for the input data to be in a predetermined syntax appropriate for application of the generative model 1060. In the case where the generative model 1060 is in the form of a large language model, the predetermined syntax is similar to the example shown in FIG. 3. Accordingly, the input data received in step S400 of FIG. 4 may be in a similar form as the data in FIG. 3. Alternatively, the method of FIG. 4 may include an intermediate step between steps S400 and S402 of converting or transforming the received input data. This may be achieved in the same manner as for the raw training data if the raw input data is in the form of tabular data, or the like.

Specifically, the conversion may comprise the following steps:
1. Extract data relating to static (e.g. date of birth) attributes and data relating to longitudinal attributes (e.g. heart rate measurement on 05.05.2023) data.
2. For longitudinal data, execute the following steps for each day where a measurement has been taken:
   i. Convert absolute date to the relative data from the previous measurement (e.g. if previous measurement was on 01.05.2023 and the current measurement is on 05.05.2023→convert to “4 days later”). If it is the first measurement, use “0 days later”.
   ii. For every measurement, convert the measurement name into a unique, descriptive name. This may be performed using a lookup table, which may be manually generated.
3. Append data relating to static attributes (alternatively referred to as “baseline data”), converting the measurement names to a unique, descriptive name, in the same manner as above.
4. Append data defining the desired output:
   i. List of attributes whose values are to be predicted.
   ii. Time frame over which the values are to be predicted.

In some cases, all instances of punctuation marks such as quotation marks (“) may also be removed, in order to reduce the computational load on the large language model.

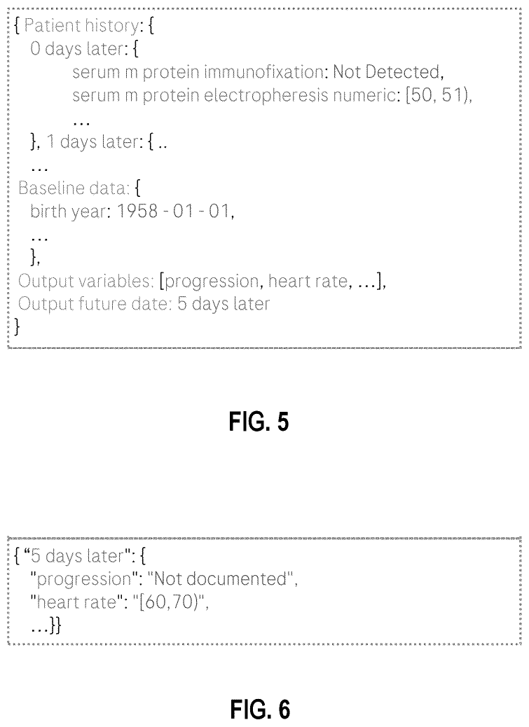

FIG. 5 is an example of input data generated using the above algorithm. It will be appreciated that the form of the input data is very similar to the training data generated in the same way. Accordingly, the JSON object is separated into patient history, which contains data describing longitudinal clinical variables, and baseline data, which contains data describing static variables. Within the patient history, each set of clinical data is divided into a different time frame, including data from “0 days later” (i.e. the first measurements) and data from “1 days later”. Within each time frame, the values of various subject-related attributes are specified, including serum m protein immunofixation and serum m protein electrophoresis numeric. Within the baseline data, there are various attributes including birth year. In addition, the input data also includes output variables including progression and heart rate, and an output future date which, again expressed in relative terms is 5 days later. These represent the time frame and the subject-related attributes which are to be output by the generative machine-learning model. By expressing the input data in the same syntax as the training data, the accuracy of the output can be improved.

Returning to FIG. 4, now that the input data has been received, and optionally converted, as outlined above, it may be stored in the buffer 1064 of the memory 106 of the forecasting system 100. In step S402 of FIG. 4, the generative machine-learning model 1062 is retrieved from the memory 106 of the forecasting module 100. Then, the initialization sub-module 10420 of the forecasting module 1042 of the processor 104 of the forecasting system 100 initializes the retrieved generative machine-learning model 1062 by inputting the input data into the generative machine-learning model 1062. Then, the generative model application sub-module 10422 runs the now-initialized generative machine-learning model 1062. In step S404, the generative machine-learning model 1062 having been run by the generative model application sub-module 10422, the output data is generated and output by the output sub-module 10424 of the forecasting module 1042 of the processor 104 of the forecasting module. In some cases, the output data may take the form shown in FIG. 6, i.e. in a JSON object. The output data may subsequently be transmitted to the display component 300 via the display component interface module 108 of the forecasting module, for display to a user. The display component 300 may be part of the client device 200.

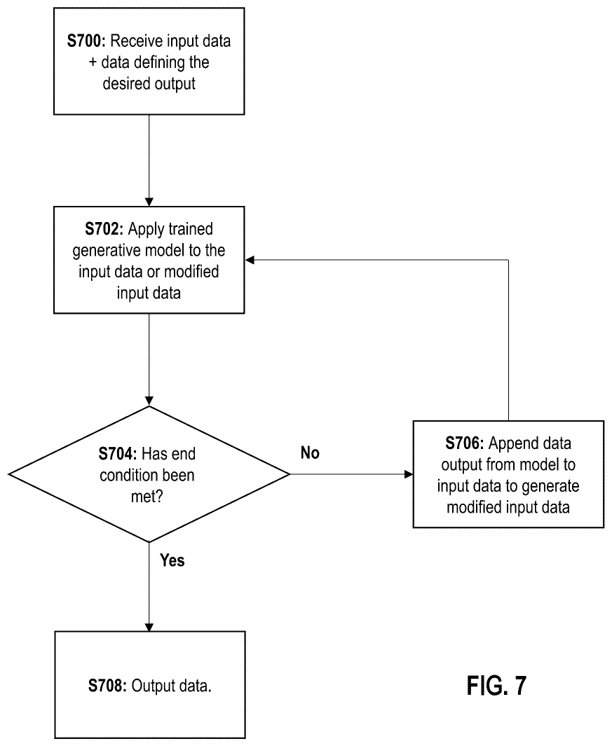

In some cases, after these values have been output, the computer-implemented method may end. However, in some cases, the computer-implemented method may be executed recursively in order to obtain a plurality of output points, rather than just a single output point (per subject-related attribute). An exemplary process is shown in FIG. 7. In step S700, the input data is received at the forecasting system 100 from the client device 200 via the client device interface module 102 of the forecasting system 100.

As before, in this step, the client device 200, more specifically the user input module 2020 of the processor 202 of the client device 200 may receive a user input. In one implementation, the user input may comprise a subject identifier, or an identifier of a medical history of a subject of interest. In response, the processor 202 may retrieve a medical history from the medical history database 2040. The request generation module 2022 of the processor 202 of the client device 200 is then configured to generate the request to be sent to the forecasting system 100. While the request is being generated by the request generation module 2022, it may be stored in the buffer 2024. After the request is generated, it may be transmitted by the transmission module 2024, whereupon it is received at the forecasting system 100 via the client device interface module 102. The input data may then be stored in buffer 1064 of the memory 106 of the forecasting system 100.

Then, in step S702, the trained generative machine-learning model 1060 is applied to the input data. More specifically, and as was the case for FIG. 4, the generative machine-learning model 1062 is retrieved from the memory 106 of the forecasting module 100. Then, the initialization sub-module 10420 of the forecasting module 1042 of the processor 104 of the forecasting system 100 initializes the retrieved generative machine-learning model 1062 by inputting the input data into the generative machine-learning model 1062. Then, the generative model application sub-module 10422 runs the now-initialized generative machine-learning model 1062, thereby generating intermediate output data. In step S704, it is determined whether an end condition is met. An example of an end condition may be that the process has been repeated a predetermined number of times. Another example of an end condition may be that output data has been generated at desired intervals for the whole of the specified time frame (e.g. output data has been generated for the next two years, with a data point being forecast for every month). Another example of an end condition may be that output data has been generated for a predetermined date. If it is determined in step S704 that the end condition has been met, the process proceeds to step S708, where the output data is output by the output sub-module 10424 of the forecasting module 1042 of the processor 104 of the forecasting module. In some cases, the output data may take the form shown in FIG. 6, i.e. in a JSON object. The output data may subsequently be transmitted to the display component 300 via the display component interface module 108 of the forecasting module, for display to a user. The display component 300 may be part of the client device 200, as discussed.

If it is determined that the end condition has not (yet) been met, the process proceeds to step S706, in which the intermediate output data is appended to the input data to generate modified input data. For example, the output data as shown in FIG. 6 may be incorporated into the input data as shown in FIG. 5, by adding an additional element to the JSON object representing the input data corresponding to the date represented by the intermediate output data. After this, the process returns to step S702 in which the trained generative machine-learning model 1062 is applied the modified input data. It will be appreciated that by virtue of the condition in step S704, the process repeats iteratively, or recursively, as necessary until the end condition is met, at which point the data is output.

The output data may be in the form of a single data point corresponding only to the most recent intermediate output data, or a series of data points may be output, representing a trajectory comprising all of the intermediate output data points.

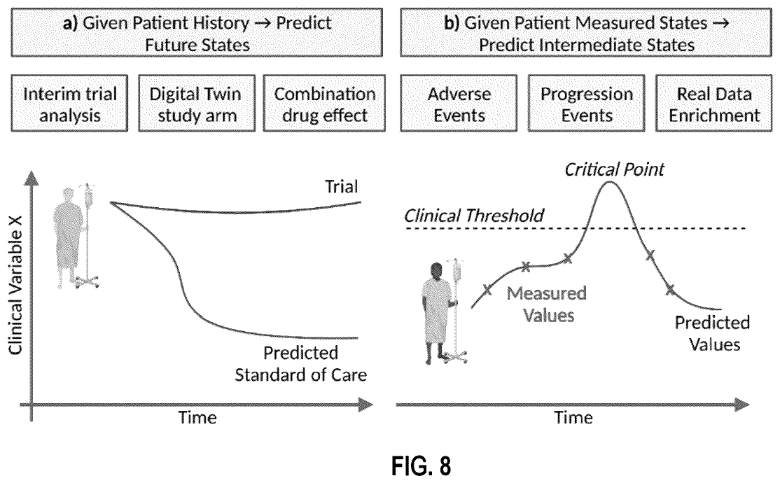

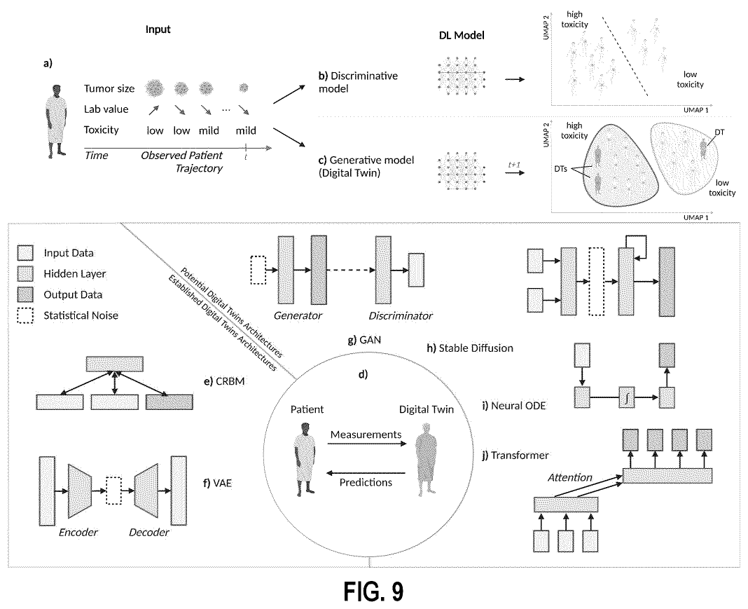

FIG. 8 sets out various use cases of implementations of the present invention. Naturally, this is not an exhaustive list:
a) In a first use case, given a patient history (i.e. a medical history), the process of the present invention may be used to determine future states. Three examples are given.
   i. This may be used for interim trial analysis. In other words, at an intermediate stage of a clinical trial, intermediate results may be complied for a given patient. These intermediate results may form the medical history in the input data. Then, by applying the generative machine-learning model to a medical history comprising these intermediate results, the output data may represent an expected trajectory for one or more variables if the subject continues with the clinical trial. In these cases, an indication of the therapeutic indication may be included in the data specifying the desired output. However, given that it is unlikely that data corresponding to the trial therapeutic intervention will have been obtained in large enough volumes to form meaningful training data, the method may simply rely on the measurements obtained during the clinical trial to forecast the trajectory. The computer-implemented method may further comprise determining whether to continue with a clinical trial based on the forecast output data. The computer-implemented method may further comprise detecting that a value of a subject-related attribute exceeds or falls below a safety threshold, and generating an alert in response to the detection. In response to the alert, the computer-implemented invention may comprise generating an output instructing a user to halt the clinical trial.
   ii. As discussed elsewhere, the present invention may be used to represent a digital twin study arm, i.e. a control arm. We will not repeat this discussion here.
   iii. Similarly, the present invention may also be used to investigate combination therapies. In particular, given clinical trial results relating to a combination therapy including a first therapeutic intervention and a second therapeutic intervention, the computer-implemented method of the present invention may be used to predict an expected trajectory of various subject-related attributes (i.e. a response to the first therapeutic intervention and/or the second therapeutic intervention), and to compare these with the results from the clinical trial in order to establish the effect of the combination therapy, as compared to the first therapeutic intervention and/or second therapeutic intervention alone. In these cases, the computer-implemented method may further comprise a step of determining a value of an efficacy metric indicative of the efficacy of the combination therapy (e.g. as compared to either therapeutic intervention alone) based on the comparison(s). The computer-implemented method may further comprise selecting a combination therapy for further investigation based on the determined value of the efficacy metric.
b) In a second use case, given a set of measurements, the present invention may be used to predict intermediate states. This may be achieved by appropriate selection of the time frame.
   i. This may be done to identify whether any adverse conditions are likely to have occurred between measurements. For example, having predicted one or more intermediate data points, the computer-implemented method may further comprise determining whether the value of a given attribute has, at any point, exceeded or fallen below a safety threshold. The computer-implemented method may further comprise detecting that a value of a subject-related attribute exceeds or falls below a safety threshold, and generating an alert in response to the detection. In response to the alert, the computer-implemented invention may comprise generating an output instructing a user to halt a clinical trial.
   ii. Progression events. In general, progression events are when the disease worsens (for example, when the tumour grows). For example, in multiple myeloma, a progression event is characterized by (among some other variables) when a specific blood value (m protein) goes above a measurable threshold. So if we can predict intermediate values that went over what we consider measurable, we could pick up on a disease progression which would have been missed in other cases. Disease progression is important for clinical trials, as they often use it for efficacy measurement
   iii. The present invention may predict intermediate values to enrich available data. This may be useful, for example, to supplement or augment training data for another machine-learning model.

Another use case (not shown) is to generate synthetic data, which is effectively anonymized, and therefore can be used for subsequent analysis or training of other machine-learning models.

### General Statements

The features disclosed in the foregoing description, or in the following claims, or in the accompanying drawings, expressed in their specific forms or in terms of a means for performing the disclosed function, or a method or process for obtaining the disclosed results, as appropriate, may, separately, or in any combination of such features, be utilised for realising the invention in diverse forms thereof.

While the invention has been described in conjunction with the exemplary embodiments described above, many equivalent modifications and variations will be apparent to those skilled in the art when given this disclosure. Accordingly, the exemplary embodiments of the invention set forth above are considered to be illustrative and not limiting. Various changes to the described embodiments may be made without departing from the spirit and scope of the invention.

For the avoidance of any doubt, any theoretical explanations provided herein are provided for the purposes of improving the understanding of a reader. The inventors do not wish to be bound by any of these theoretical explanations.

Any section headings used herein are for organizational purposes only and are not to be construed as limiting the subject matter described.

Throughout this specification, including the claims which follow, unless the context requires otherwise, the word “comprise” and “include”, and variations such as “comprises”, “comprising”, and “including” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment. The term “about” in relation to a numerical value is optional and means for example +/−10%.

### REFERENCES

[1] Wong C H, Siah K W, Lo A W. Estimation of clinical trial success rates and related parameters. Biostatistics. 2019 Apr. 1; 20(2):273-86.
[2] Friedman L M, Furberg C D, DeMets D L, Reboussin D M, Granger C B. Fundamentals of Clinical Trials. Cham (Switzerland): Springer International Publishing; 2015.
[3] Brøgger-Mikkelsen M, Ali Z, Zibert J R, Andersen A D, Thomsen S F. Online Patient Recruitment in Clinical Trials: Systematic Review and Meta-Analysis. Journal of Medical Internet Research. 2020 Nov. 4; 22(11):e22179.
[4] Kamel Boulos M N, Zhang P. Digital Twins: From Personalised Medicine to Precision Public Health. Journal of Personalized Medicine. 2021 August; 11(8):745.
[5] Armeni P, Polat I, De Rossi L M, Diaferia L, Meregalli S, Gatti A. Digital Twins in Healthcare: Is It the Beginning of a New Era of Evidence-Based Medicine? A Critical Review. Journal of Personalized Medicine. 2022 August; 12(8):1255.
[6] Woodcock J, LaVange L M. Master Protocols to Study Multiple Therapies, Multiple Diseases, or Both. New England Journal of Medicine. 2017 Jul. 6; 377(1):62-70.
[7] Susilo M E, Li C C, Gadkar K, Hernandez G, Huw L Y, Jin J Y, Yin S, Wei M C, Ramanujan S, Hosseini I. Systems-based Digital Twins to Help Characterize Clinical Dose-Response and Propose Predictive Biomarkers in a Phase I Study of Bispecific Antibody, Mosunetuzumab, in NHL. Clinical and Translational Science. 2023 Mar. 13.
[8] Kaul R, Ossai C, Forkan A R M, Jayaraman P P, Zelcer J, Vaughan S, et al. The role of AI for developing digital twins in healthcare: The case of cancer care. WIREs Data Mining and Knowledge Discovery. 2023; 13(1):e1480.
[9] Dhillon A, Singh A. Machine learning in healthcare data analysis: a survey. Journal of Biology and Today's World. 2019; 8(6):1-0.
[10] Croitoru F A, Hondru V, Ionescu R T, Shah M. Diffusion Models in Vision: A Survey. IEEE Transactions on Pattern Analysis and Machine Intelligence. 2023; 1-20.
[11] Lin T, Wang Y, Liu X, Qiu X. A survey of transformers. AI Open. 2022 Jan. 1; 3:111-32.
[12] Chen R T, Rubanova Y, Bettencourt J, Duvenaud D K. Neural ordinary differential equations. Advances in neural information processing systems. 2018; 31.
[13] Mak K K, Pichika M R. Artificial intelligence in drug development: present status and future prospects. Drug Discovery Today. 2019 Mar. 1; 24(3):773-80.
[14] Weissler E H, Naumann T, Andersson T, Ranganath R, Elemento O, Luo Y, et al. The role of machine learning in clinical research: transforming the future of evidence generation. Trials. 2021 Aug. 16; 22(1):537.
[15] 1. Lee G, Kang B, Nho K, Sohn K A, Kim D. MildInt: Deep Learning-Based Multimodal Longitudinal Data Integration Framework. Frontiers in Genetics. 2019; 10.
[16] Bertolini D, Loukianov A D, Smith A M, Li-Bland D, Pouliot Y, Walsh J R, Fisher C K. Modeling Disease Progression in Mild Cognitive Impairment and Alzheimer's Disease with Digital Twins. arXiv preprint arXiv: 2012.13455. 2020 Dec. 24.
[17] Walsh J R, Smith A M, Pouliot Y, Li-Bland D, Loukianov A, Fisher C K. Generating digital twins with multiple sclerosis using probabilistic neural networks. arXiv preprint arXiv: 2002.02779. 2020 Feb. 4.
[18] Allen A, Siefkas A, Pellegrini E, Burdick H, Barnes G, Calvert J, et al. A Digital Twins Machine Learning Model for Forecasting Disease Progression in Stroke Patients. Applied Sciences. 2021 January; 11(12):5576.
[19] Angermueller C, Pärnamaa T, Parts L, Stegle O. Deep learning for computational biology. Mol Syst Biol. 2016 Jul. 29; 12(7):878.
[20] Walsh J R, Roumpanis S, Bertolini D, Delmar P. Evaluating Digital Twins for Alzheimer's Disease using Data from a Completed Phase 2 Clinical Trial. Alzheimer's & Dementia. 2022; 18(S10):e065386.
[21] Beaulieu-Jones B K, Wu Z S, Williams C, Lee R, Bhavnani S P, Byrd J B, et al. Privacy-Preserving Generative Deep Neural Networks Support Clinical Data Sharing. Circulation: Cardiovascular Quality and Outcomes. 2019 July; 12(7):e005122.
[22] Qualification opinion for Prognostic Covariate Adjustment (PROCOVA™) [Internet], Committee for Medicinal Products for Human Use (CHMP); 2022 Sep. 15 [cited 2023 Jun. 1]. Available from https://www.ema.europa.eu/en/documents/regulatory-procedural-guideline/qualification-opinion-prognostic-covariate-adjustment-procovatm_en.pdf
[23] Raffel C, Shazeer N, Roberts A, Lee K, Narang S, Matena M, Zhou Y, Li W, Liu P J. Exploring the limits of transfer learning with a unified text-to-text transformer. The Journal of Machine Learning Research. 2020 Jan. 1; 21(1):5485-551. (https://jmlr.org/papers/volume21/20-074/20-074.pdf)
[24] Guo M, Ainslie J, Uthus D, Ontanon S, Ni J, Sung Y H, Yang Y. LongT5: Efficient text-to-text transformer for long sequences. arXiv preprint arXiv: 2112.07916. 2021 Dec. 15. (https://arxiv.org/abs/2112.07916)
[25] https://www.mosaicml.com/blog/mpt-7b; https://huggingface.co/mosaicml/mpt-7b
[26] Phang J, Zhao Y, Liu P J. Investigating efficiently extending transformers for long input summarization. arXiv preprint arXiv: 2208.04347. 2022 Aug. 8. (https://arxiv.org/abs/2208.04347)
[27] Beltagy I, Peters M E, Cohan A. Longformer: The long-document transformer. arXiv preprint arXiv: 2004.05150. 2020 Apr. 10. (https://arxiv.org/abs/2004.05150)
[28] Radford A, Narasimhan K, Salimans T, Sutskever I. Improving language understanding by generative pre-training. (https://s3-us-west-2.amazonaws.com/openai-assets/research-covers/language-unsupervised/language_understanding_paper.pdf)
[29] Radford A, Wu J, Child R, Luan D, Amodei D, Sutskever I. Language models are unsupervised multitask learners. OpenAI blog. 2019 Feb. 24; 1(8):9. (https://d4mucfpksywv.cloudfront.net/better-language-models/language_models_are_unsupervised_multitask_learners.pdf)
[30] Brown T, Mann B, Ryder N, Subbiah M, Kaplan J D, Dhariwal P, Neelakantan A, Shyam P, Sastry G, Askell A, Agarwal S. Language models are few-shot learners. Advances in neural information processing systems. 2020; 33:1877-901. (https://arxiv.org/abs/2005.14165)
[31] https://openai.com/blog/chatgpt
[32] https://openai.com/research/gpt-4; https://arxiv.org/abs/2303.08774
[33] Poli M, Massaroli S, Nguyen E, Fu D Y, Dao T, Baccus S, Bengio Y, Ermon S, Ré C. Hyena hierarchy: Towards larger convolutional language models. arXiv preprint arXiv: 2302.10866. 2023 Feb. 21. (https://arxiv.org/abs/2302.10866)
[34] Touvron H, Lavril T, Izacard G, Martinet X, Lachaux M A, Lacroix T, Rozière B, Goyal N, Hambro E, Azhar F, Rodriguez A. Llama: Open and efficient foundation language models. arXiv preprint arXiv: 2302.13971. 2023 Feb. 27 (https://arxiv.org/abs/2302.13971)
[35] https://falconllm.tii.ae/; https://huggingface.co/tiiuae/falcon-40b

ANNEX - List of subject-related attributes

| | |
|---|---|
| Serum calcium | (ionized) |
| Serum calcium | (blood, ionized) |
| Serum calcium | (mass to volume, blood) |
| Serum calcium | ionized, ion-selective membrane electrode) |
| Serum calcium | moles to volume |
| Haemoglobin | (a1c to hemoglobin total) |
| Haemoglobin | by calculation |
| Serum creatinine | (mass to volume in blood |
| Serum FLC | kappa light chains/lambda light chains [mass ratio] in urine |
| Serum FLC | kappa light chains.free/lambda light chains.free [mass ratio] in |
| | 24 hour urine |
| Serum FLC | kappa light chains.free [mass/volume] in urine |
| Serum FLC | lambda light chains.free [mass/volume] in urine |
| Serum FLC | kappa light chains.free [mass/time] in 24 hour urine |
| Serum FLC | lambda light chains.free [mass/time] in 24 hour urine |
| Serum FLC | lambda light chains.free [mass/volume] in 24 hour urine |
| Serum FLC | kappa light chains.free [mass/volume] in 24 hour urine |
| Serum FLC | Kappa light chains/Lambda light chains |
| General | Immunofixation for Serum or Plasma |
| M Protein | igg [mass/volume] in serum or plasma |
| General | |
| M Protein | iga [mass/volume] in serum or plasma |
| General | |
| M Protein | igm [mass/volume] in serum or plasma |
| General | |
| M Protein | igd [mass/volume] in serum |
| General | |
| M Protein | ige [units/volume] in serum or plasma |
| General | |
| Inclusion | bilirubin.total [mass/volume] in serum or plasma |
| Criteria | |
| Inclusion | aspartate aminotransferase [enzymatic activity/volume] in |
| Criteria | serum or plasma |
| Inclusion | alanine aminotransferase [enzymatic activity/volume] in serum |
| Criteria | or plasma |
| Inclusion | platelets [#/volume] in blood |
| Criteria | |
| Inclusion | creatinine renal clearance predicted by cockcroft-gault formula |
| Criteria | |

body height
heart rate
body weight
ecog
diastolic blood pressure
systolic blood pressure
body temperature
oxygen saturation in arterial blood by pulse oximetry
pain severity - 0-10 verbal numeric rating [score] - reported
respiratory rate
body surface area
hemoglobin [mass/volume] in blood
urea nitrogen [mass/volume] in serum or plasma
calcium [mass/volume] in serum or plasma
creatinine [mass/volume] in serum or plasma
protein [mass/volume] in serum or plasma
alkaline phosphatase [enzymatic activity/volume] in serum or plasma
aspartate aminotransferase [enzymatic activity/volume] in serum or plasma
alanine aminotransferase [enzymatic activity/volume] in serum or plasma
albumin [mass/volume] in serum or plasma
bilirubin.total [mass/volume] in serum or plasma
carbon dioxide, total [moles/volume] in serum or plasma
glucose [mass/volume] in serum or plasma
chloride [moles/volume] in serum or plasma
potassium [moles/volume] in serum or plasma
sodium [moles/volume] in serum or plasma
platelets [#/volume] in blood
hematocrit [volume fraction] of blood
leukocytes [#/volume] in blood
erythrocytes [#/volume] in blood
igg [mass/volume] in serum or plasma
iga [mass/volume] in serum or plasma
kappa light chains.free [mass/volume] in serum
igm [mass/volume] in serum or plasma
lambda light chains.free [mass/volume] in serum or plasma
lymphocytes/100 leukocytes in blood
lymphocytes [#/volume] in blood
monocytes/100 leukocytes in blood
monocytes [#/volume] in blood
neutrophils [#/volume] in blood
eosinophils [#/volume] in blood
basophils [#/volume] in blood
eosinophils/100 leukocytes in blood
basophils/100 leukocytes in blood
beta-2-microglobulin [mass/volume] in serum or plasma
glomerular filtration rate/1.73 sq m.predicted among blacks [volume rate/area]
in serum, plasma or blood by creatinine-based formula (mdrd)
kappa light chains.free/lambda light chains.free [mass ratio] in serum
albumin [mass/volume] in serum or plasma by electrophoresis
glomerular filtration rate/1.73 sq m.predicted among non-blacks [volume
rate/area] in serum, plasma or blood by creatinine-based formula (mdrd)
ferritin [mass/volume] in serum or plasma
neutrophils/100 leukocytes in blood
glomerular filtration rate/1.73 sq m.predicted [volume rate/area] in serum,
plasma or blood
magnesium [mass/volume] in serum or plasma
protein [mass/volume] in urine
immunofixation for serum or plasma
lactate dehydrogenase [enzymatic activity/volume] in serum or plasma
granulocytes [#/volume] in blood
granulocytes/100 leukocytes in blood
thyrotropin [units/volume] in serum or plasma
protein.monoclonal [mass/volume] in serum or plasma by electrophoresis
kappa light chains/lambda light chains [mass ratio] in serum
inr in platelet poor plasma or blood by coagulation assay
prothrombin time (pt)
protein [mass/time] in 24 hour urine
lymphocytes [#/volume] in blood by automated count
monocytes [#/volume] in blood by automated count
lymphocytes/100 leukocytes in blood by automated count
monocytes/100 leukocytes in blood by automated count
basophils [#/volume] in blood by automated count
leukocytes [#/volume] in blood by automated count
erythrocytes [#/volume] in blood by automated count
basophils/100 leukocytes in blood by automated count
albumin/protein.total in urine by electrophoresis
hematocrit [volume fraction] of blood by automated count
platelets [#/volume] in blood by automated count
aptt in platelet poor plasma by coagulation assay
neutrophils [#/volume] in blood by automated count
lymphocytes/100 leukocytes in blood by manual count
monocytes/100 leukocytes in blood by manual count
bilirubin.direct [mass/volume] in serum or plasma
eosinophils/100 leukocytes in blood by manual count
neutrophils/100 leukocytes in blood by automated count
immunofixation for urine
monocytes [#/volume] in blood by manual count
lymphocytes [#/volume] in blood by manual count
gamma globulin/protein.total by electrophoresis in urine collected for
unspecified duration
eosinophils [#/volume] in blood by manual count
band form neutrophils/100 leukocytes in blood by manual count
basophils/100 leukocytes in blood by manual count
creatinine [mass/volume] in urine
basophils [#/volume] in blood by manual count
lactate dehydrogenase [enzymatic activity/volume] in serum or plasma by
lactate to pyruvate reaction
neutrophils [#/volume] in blood by manual count
band form neutrophils [#/volume] in blood
protein.monoclonal band 1 [mass/volume] in serum or plasma by
electrophoresis
segmented neutrophils/100 leukocytes in blood by manual count
erythrocyte sedimentation rate
bilirubin.indirect [mass/volume] in serum or plasma
creatinine [mass/time] in 24 hour urine
cholesterol in ldl [mass/volume] in serum or plasma by direct assay
protein.monoclonal [mass/time] in 24 hour urine by electrophoresis
beta-2-microglobulin ser/plas mcnc pt qn
albumin ser/plas mcnc pt qn
urate [mass/volume] in serum or plasma
platelets [#/volume] in blood by estimate
c reactive protein [mass/volume] in serum or plasma
hemoglobin a1c/hemoglobin.total in blood
sodium [moles/volume] in blood
segmented neutrophils/100 leukocytes in blood
band form neutrophils/100 leukocytes in blood
protein [mass/volume] in 24 hour urine
segmented neutrophils [#/volume] in blood
granulocytes [#/volume] in blood by automated count
potassium [moles/volume] in blood
creatinine renal clearance predicted by cockcroft-gault formula
kappa light chains.free [mass/volume] in urine
granulocytes/100 leukocytes in blood by automated count
protein.monoclonal/protein.total in 24 hour urine by electrophoresis
thyroxine (t4) free [mass/volume] in serum or plasma
lambda light chains.free [mass/volume] in urine
erythropoietin (epo) [units/volume] in serum or plasma
protein.monoclonal/protein.total in urine by electrophoresis
thyroxine (t4) [mass/volume] in serum or plasma
creatinine renal clearance in urine and serum or plasma collected for
unspecified duration
kappa light chains [mass/volume] in serum or plasma
prostate specific ag [mass/volume] in serum or plasma
calcium.ionized [moles/volume] in blood
albumin/protein.total in serum or plasma
erythrocyte sedimentation rate by westergren method
lactate dehydrogenase ser/plas ccnc pt qn
protein [mass/volume] in urine collected for unspecified duration
lambda light chains [mass/volume] in serum or plasma
hepatitis b virus surface ag [presence] in serum
gamma glutamyl transferase [enzymatic activity/volume] in serum or plasma
kappa light chains.free/lambda light chains.free [mass ratio] in urine
protein.monoclonal band 2 [mass/volume] in serum or plasma by
electrophoresis
ige [units/volume] in serum or plasma
creatinine [mass/volume] in blood
albumin/protein.total by electrophoresis in urine collected for unspecified
duration
c reactive protein [mass/volume] in serum or plasma by high sensitivity
method
hepatitis b virus core ab [presence] in serum
blasts/100 leukocytes in blood
albumin/protein.total in serum or plasma by electrophoresis
fibrin d-dimer feu [mass/volume] in platelet poor plasma
carcinoembryonic ag [mass/volume] in serum or plasma
hepatitis b virus surface ab [units/volume] in serum
creatinine renal clearance/1.73 sq m in urine and serum or plasma collected
for unspecified duration
albumin [mass/volume] in urine by electrophoresis
thyroxine (t4) free index in serum or plasma by calculation
calcium.ionized [mass/volume] in serum or plasma
protein.abnormal band [mass/time] in 24 hour urine
blasts/100 leukocytes in blood by manual count
bilirubin.conjugated [mass/volume] in serum or plasma
kappa light chains/lambda light chains [mass ratio] in urine
bicarbonate [moles/volume] in venous blood
testosterone [mass/volume] in serum or plasma
troponin i.cardiac [mass/volume] in serum or plasma
troponin t.cardiac [mass/volume] in serum or plasma
bicarbonate [moles/volume] in arterial blood
hepatitis c virus ab [presence] in serum
kappa light chains.free [mass/time] in 24 hour urine
lambda light chains.free [mass/time] in 24 hour urine
albumin [mass/time] in 24 hour urine by electrophoresis
glomerular filtration rate/1.73 sq m.predicted [volume rate/area] in serum,
plasma or blood by creatinine-based formula (ckd-epi)
kappa light chains [mass/volume] in urine
cancer related multigene analysis in blood or tissue by molecular genetics
method
troponin i.cardiac [mass/volume] in blood
hepatitis c virus ab signal/cutoff in serum or plasma by immunoassay
hepatitis b virus core igm ab [presence] in serum
igd [mass/volume] in serum
lambda light chains [mass/volume] in urine
blasts [#/volume] in blood
protein.monoclonal/protein.total in serum or plasma by electrophoresis
hepatitis b virus surface ab [presence] in serum
calcium.ionized [moles/volume] in serum or plasma
troponin t.cardiac [mass/volume] in blood
glomerular filtration rate/1.73 sq m.predicted [volume rate/area] in serum,
plasma or blood by creatinine-based formula (ckd-epi 2021)
kappa light chains [mass/time] in 24 hour urine
cortisol [mass/volume] in serum or plasma
protein.monoclonal band 3 [mass/volume] in serum or plasma by
electrophoresis
protein.monoclonal [mass/volume] in urine by electrophoresis
follitropin [units/volume] in serum or plasma
cancer ag 19-9 [units/volume] in serum or plasma
granulocytes [#/volume] in blood by manual count
calcium.ionized [mass/volume] in blood
platelets [#/volume] in blood by manual count
microalbumin [mass/volume] in urine
lutropin [units/volume] in serum or plasma
bicarbonate [moles/volume] in serum or plasma
albumin [mass/volume] in urine
hepatitis c virus ab [presence] in serum or plasma by immunoassay
lipase [enzymatic activity/volume] in serum or plasma
cancer ag 27-29 [units/volume] in serum or plasma
hepatitis c virus ab [units/volume] in serum
protein.monoclonal [mass/volume] in urine
band form neutrophils [#/volume] in blood by automated count
hepatitis c virus rna [units/volume] (viral load) in serum or plasma by naa with
probe detection
amylase [enzymatic activity/volume] in serum, plasma or blood
bicarbonate [moles/volume] in blood
cardiolipin igg ab [units/volume] in serum or plasma
cardiolipin igm ab [units/volume] in serum or plasma
kappa light chains.free/lambda light chains.free [mass ratio] in 24 hour urine
protein.abnormal band [mass/volume] in serum
prostate specific ag free [mass/volume] in serum or plasma
albumin [mass/time] in 24 hour urine
albumin [presence] in 24 hour urine by electrophoresis
cancer ag 15-3 [units/volume] in serum or plasma
prostate specific ag free/prostate specific ag.total in serum or plasma
kappa light chains/lambda light chains [mass ratio] in 24 hour urine
alpha-1-fetoprotein.tumor marker [mass/volume] in serum or plasma
lambda light chains.free [mass/volume] in 24 hour urine
cardiolipin iga ab [units/volume] in serum or plasma
hepatitis c virus rna [log units/volume] (viral load) in serum or plasma by naa
with probe detection
albumin [mass/volume] in serum or plasma by bromocresol green (bcg) dye
binding method
blasts [#/volume] in blood by manual count
corticotropin [mass/volume] in plasma
prolactin [mass/volume] in serum or plasma
albumin [presence] in urine
calcium [mass/volume] in blood
glomerular filtration rate/1.73 sq m.predicted among blacks [volume rate/area]
in serum, plasma or blood by creatinine-based formula (ckd-epi)
fasting glucose [mass/volume] in serum or plasma
glomerular filtration rate/1.73 sq m.predicted among non-blacks [volume
rate/area] in serum, plasma or blood by creatinine-based formula (ckd-epi)
kappa light chains.free [mass/volume] in 24 hour urine
hepatitis c virus ab [units/volume] in serum by immunoassay
beta-2-microglobulin [mass/volume] in urine
glomerular filtration rate/1.73 sq m.predicted among females [volume
rate/area] in serum, plasma or blood by creatinine-based formula (mdrd)
alanine aminotransferase [enzymatic activity/volume] in serum or plasma by
with p-5′-p
immunoglobulin light chains [mass/time] in 24 hour urine
microalbumin [mass/volume] in urine by detection limit <=1.0 mg/l
hemoglobin [mass/volume] in blood by calculation
hepatitis b virus core ab [units/volume] in serum by immunoassay
prostate specific ag.free ser/plas mcnc pt qn
aspartate aminotransferase [enzymatic activity/volume] in serum or plasma by
with p-5′-p
cortisol [mass/volume] in serum or plasma --am peak specimen
protein.monoclonal [mass/volume] in 24 hour urine by electrophoresis
chromogranin a [mass/volume] in serum or plasma
alpha-1-fetoprotein [mass/volume] in serum or plasma
hepatitis b virus surface ag [units/volume] in serum
microalbumin [mass/volume] in 24 hour urine
prealbumin [mass/volume] in serum or plasma
5-hydroxyindoleacetate [mass/time] in 24 hour urine
urate [mass/volume] in urine
band form neutrophils/100 leukocytes in blood by automated count
cancer ag 125 [units/volume] in serum or plasma
hepatitis c virus rna [presence] in serum or plasma by naa with probe
detection
urate [mass/time] in 24 hour urine
renin [enzymatic activity/volume] in plasma
5-hydroxyindoleacetate [mass/volume] in urine
alpha-1-fetoprotein.tumor marker [units/volume] in serum or plasma
immunoglobulin light chains [interpretation] in urine
hepatitis b virus core ab [units/volume] in serum
aldosterone [mass/volume] in serum or plasma
erythrocyte sedimentation rate by wintrobe method
glomerular filtration rate/1.73 sq m.predicted [volume rate/area] in serum,
plasma or blood by creatinine-based formula (mdrd)
hepatitis b virus surface ag [presence] in serum, plasma or blood by rapid
immunoassay
prostate specific ag [mass/volume] in serum or plasma by detection
limit <=0.01 ng/ml
progesterone [mass/volume] in serum or plasma
calcium [moles/volume] in serum or plasma
urate [mass/volume] in 24 hour urine
cortisol [mass/volume] in serum or plasma --1 hour post xxx challenge
hepatitis b virus core ab [presence] in serum or plasma by immunoassay
human papilloma virus 16 + 18 + 31 + 33 + 35 + 39 + 45 + 51 + 52 + 56 + 58 + 59 + 68 dna
[presence] in specimen by naa with probe detection
cortisol [mass/volume] in serum or plasma --30 minutes post xxx challenge
cortisol free [mass/volume] in serum or plasma
fibrin d-dimer ddu [mass/volume] in platelet poor plasma
hepatitis b virus surface ag [presence] in serum or plasma by confirmatory
method
protein.monoclonal band 1/protein.total in serum or plasma by electrophoresis
calcium.ionized [mass/volume] in serum or plasma by ion-selective
membrane electrode (ise)
chromogranin a [moles/volume] in serum or plasma
iga [units/volume] in serum
alanine aminotransferase [enzymatic activity/volume] in serum or plasma by
no addition of p-5′-p
aldosterone/renin [ratio] in plasma
cortisol [mass/volume] in serum or plasma --1 hour post dose corticotropin
cortisol free [mass/time] in 24 hour urine
5-hydroxyindoleacetate/creatinine [mass ratio] in urine
cardiolipin iga ab [presence] in serum
cortisol free [mass/volume] in urine
cortisol free/creatinine [mass ratio] in urine
hepatitis c virus rna [#/volume] (viral load) in serum or plasma by naa with
probe detection
magnesium [mass/volume] in blood
carcinoembryonic ag ser/plas mcnc pt qn
cortisol [mass/volume] in serum or plasma --30 minutes post dose
corticotropin
hepatitis b virus core igg + igm ab [presence] in serum
hepatitis b virus core igm ab [presence] in serum or plasma by immunoassay
somatotropin [mass/volume] in serum or plasma
troponin i.cardiac [presence] in serum, plasma or blood by rapid
immunoassay
bilirubin.total [mass/volume] in blood
cardiolipin igg ab [presence] in serum
enolase.neuron specific [mass/volume] in serum or plasma
hepatitis b virus surface ab [units/volume] in serum by radioimmunoassay (ria)
human papilloma virus 16 + 18 + 31 + 33 + 35 + 39 + 45 + 51 + 52 + 56 + 58 + 59 + 68 dna
[presence] in cervix by probe with signal amplification
protein.abnormal band/protein.total in urine by electrophoresis
cardiolipin igm ab [presence] in serum by immunoassay
cortisol [mass/volume] in serum or plasma --pm trough specimen
cortisol [mass/volume] in serum or plasma --pre dose corticotropin
hepatitis b virus surface ab [units/volume] in serum or plasma by
immunoassay
troponin t.cardiac [presence] in blood
alpha-1-fetoprotein [units/volume] in serum or plasma
protein.monoclonal band 2/protein.total in serum or plasma by electrophoresis
troponin t.cardiac [presence] in serum or plasma
cancer ag 19-9 ser/plas acnc pt qn
hepatitis b virus surface ag [presence] in serum or plasma by immunoassay
renin [mass/volume] in plasma
vasopressin [mass/volume] in serum or plasma
acarboxyprothrombin [mass/volume] in serum or plasma
aldosterone [mass/time] in 24 hour urine
alpha-1-fetoprotein l3/alpha-1-fetoprotein.total in serum or plasma
c reactive protein [presence] in serum or plasma
c reactive protein [quintile] in serum or plasma by high sensitivity method
cancer ag 125 ser/plas acnc pt qn
cardiolipin ab [presence] in serum
cortisol [mass/volume] in saliva (oral fluid)
cortisol/creatinine [mass ratio] in urine
creatinine ser/plas mcnc pt qn
ferritin [mass/volume] in blood
hepatitis b virus surface ag [units/volume] in serum or plasma by
immunoassay
human papilloma virus 16 ag [presence] in specimen
human papilloma virus 18 ag [presence] in specimen
lymphocytes [#/volume] in blood by flow cytometry (fc)
magnesium ionized [moles/volume] in serum or plasma
ugt1a1 gene targeted mutation analysis in blood or tissue by molecular
genetics method
Multiple myeloma not having achieved remission
Other long term (current) drug therapy
Essential (primary) hypertension
Encounter for antineoplastic chemotherapy
Multiple myeloma in remission
Stem cells transplant status
Anemia, unspecified
Multiple myeloma in relapse
Long term (current) use of opiate analgesic
Long term (current) use of oral hypoglycemic drugs
Monoclonal gammopathy
Gastro-esophageal reflux disease without esophagitis
Other fatigue
Other activity involving computer technology and electronic devices
Encounter for follow-up examination after completed treatment for conditions
other than malignant neoplasm
Anemia due to antineoplastic chemotherapy
Personal history of nicotine dependence
Encounter for immunization
Polyneuropathy, unspecified
Neoplasm related pain (acute) (chronic)
Adverse effect of antineoplastic and immunosuppressive drugs, initial
encounter
Long term (current) use of anticoagulants
Other activity involving ice and snow
Disorder of bone, unspecified
Secondary malignant neoplasm of bone
Diarrhea, unspecified
Chronic kidney disease, unspecified
Long term (current) use of aspirin
Unspecified atrial fibrillation
Encounter for antineoplastic immunotherapy
Thrombocytopenia, unspecified
Personal history of antineoplastic chemotherapy
Other joint disorder, not elsewhere classified
Dorsalgia, unspecified
Nausea
Hypertensive crisis, unspecified
Other and unspecified soft tissue disorders, not elsewhere classified
Other venous embolism and thrombosis
Atherosclerotic heart disease of native coronary artery without angina
pectoris
Acute kidney failure, unspecified
Low back pain
Other secondary thrombocytopenia
Drug-induced polyneuropathy
Hypercalcemia
Nausea with vomiting, unspecified
Anxiety disorder, unspecified
Anemia in chronic kidney disease
Anemia in neoplastic disease
Major depressive disorder, single episode, unspecified
Cough
Encounter for other preprocedural examination
Heart failure
Encounter for examination for normal comparison and control in clinical
research program
Other chronic pain
Constipation, unspecified
Body mass index [BMI]
Insomnia, unspecified
Personal history of irradiation
Localized edema
Nonfamilial hypogammaglobulinemia
Weakness
Neutropenia, unspecified
Long term (current) use of bisphosphonates
Other pancytopenia
Agranulocytosis secondary to cancer chemotherapy
Iron deficiency anemia, unspecified
Personal history of malignant neoplasm
Shortness of breath
Unspecified lump in breast
Hypomagnesemia
Pure hypercholesterolemia, unspecified
Personal history of other venous thrombosis and embolism
Chronic kidney disease, stage 3 (moderate)
Antineoplastic chemotherapy induced pancytopenia
Hypertensive chronic kidney disease with stage 1 through stage 4 chronic
kidney disease, or unspecified chronic kidney disease
Disorder of continuity of bone
Other spondylopathies
Pain, unspecified
Disturbances of skin sensation
Encounter for general adult medical examination without abnormal findings
Long term (current) use of insulin
Fracture at wrist and hand level
Fracture of rib(s), sternum and thoracic spine
Other malaise
Dorsalgia
Unspecified osteoarthritis, unspecified site
Disorder of kidney and ureter, unspecified
Adverse effect of antineoplastic and immunosuppressive drugs, subsequent
encounter
Edema, unspecified
Poisoning by, adverse effect of and underdosing of diuretics and other and
unspecified drugs, medicaments and biological substances
Acquired absence of organs, not elsewhere classified
Age-related osteoporosis without current pathological fracture
Personal history of other diseases and conditions
Benign prostatic hyperplasia without lower urinary tract symptoms
Chronic kidney disease, stage 4 (severe)
Unspecified asthma, uncomplicated
Long term (current) use of systemic steroids
Fever, unspecified
Abdominal and pelvic pain
Solitary plasmacytoma not having achieved remission
Heart failure, unspecified
Glaucoma
Other pulmonary embolism without acute cor pulmonale
Type 2 diabetes mellitus with hyperglycemia
Disorder of bone density and structure, unspecified
Urinary tract infection, site not specified
Malignant neoplasm of prostate
Fracture of lumbar spine and pelvis
Other pulmonary heart diseases
Acute embolism and thrombosis of unspecified deep veins of unspecified
lower extremity
Other cardiac arrhythmias
Disorder of cartilage, unspecified
Poisoning by, adverse effect of and underdosing of primarily systemic and
hematological agents, not elsewhere classified
Chronic obstructive pulmonary disease, unspecified
Poisoning by, adverse effect of and underdosing of psychotropic drugs, not
elsewhere classified
Rash and other nonspecific skin eruption
Thoracic, thoracolumbar, and lumbosacral intervertebral disc disorders
Encounter for adjustment and management of vascular access device
Other coagulation defects
Fracture of forearm
Family history of primary malignant neoplasm
Contact with and (suspected) exposure to other viral communicable
diseases
Decreased white blood cell count, unspecified
Paroxysmal atrial fibrillation
Obstructive sleep apnea (adult) (pediatric)
Vitamin B12 deficiency anemia
Abnormal findings on diagnostic imaging of other body structures
Pneumonia, unspecified organism
Chronic kidney disease (CKD)
Other disorders involving the immune mechanism, not elsewhere classified
Other symptoms and signs involving cognitive functions and awareness
Cardiomyopathy
Presence of cardiac and vascular implants and grafts
Other disorders of plasma-protein metabolism, not elsewhere classified
Encounter for screening for malignant neoplasms
Encounter for antineoplastic radiation therapy
Secondary malignant neoplasm of bone marrow
Long term (current) drug therapy
Abnormalities of breathing
Other nonspecific abnormal finding of lung field
Other respiratory disorders
Fracture of cervical vertebra and other parts of neck
Persons encountering health services for other counseling and medical
advice, not elsewhere classified
Spondylosis
Poisoning by, adverse effect of and underdosing of hormones and their
synthetic substitutes and antagonists, not elsewhere classified
Abnormalities of gait and mobility
Osteopathy in diseases classified elsewhere, unspecified site
Other retinal disorders
Personal history of other malignant neoplasm of skin
Headache
Cellulitis and acute lymphangitis
Presence of other functional implants
Personal history of certain other diseases
Dizziness and giddiness
Encounter for other prophylactic measures
Dyspnea, unspecified
Poisoning by, adverse effect of and underdosing of narcotics and
psychodysleptics [hallucinogens]
Encounter for screening for other diseases and disorders
Other specified abnormal findings of blood chemistry
Postviral fatigue syndrome
Nonrheumatic aortic valve disorders
Bone marrow transplant status
Encounter for other procedures for purposes other than remedying health
state
Stomatitis and related lesions
Unspecified abdominal pain
Abnormal weight loss
Hypocalcemia
Other and unspecified malignant neoplasm of skin
Chest pain, unspecified
Family history of malignant neoplasm of digestive organs
Encounter for other special examination without complaint, suspected or
reported diagnosis
Abnormal electrocardiogram [ECG] [EKG]
Localized swelling, mass and lump of skin and subcutaneous tissue
Acute upper respiratory infection, unspecified
Complications of cardiac and vascular prosthetic devices, implants and
grafts
Encounter for palliative care
Other postprocedural states
Encounter for screening mammogram for malignant neoplasm of breast
Light chain (AL) amyloidosis
Nutritional anemia, unspecified
Allergy status to drugs, medicaments and biological substances
Anorexia
Other dorsalgia
Other general symptoms and signs
Cervicalgia
Other disorders of phosphorus metabolism
Atrial fibrillation and flutter
Other specified postprocedural states
Long term (current) use of antibiotics
End stage renal disease
Pain in throat and chest
Hypotension, unspecified
Asthma
Abnormal results of function studies
Osteopathy in diseases classified elsewhere, multiple sites
Other drug-induced agranulocytosis
Personal risk factors, not elsewhere classified
Gastritis and duodenitis
Other specified noninfective gastroenteritis and colitis
Poisoning by, adverse effect of and underdosing of agents primarily affecting
the cardiovascular system
Personal history of pulmonary embolism
Reaction to severe stress, and adjustment disorders
Other disorders of white blood cells
Other disorders of bone
Bradycardia, unspecified
Sepsis, unspecified organism
Tachycardia, unspecified
Major depressive disorder, single episode
Polyuria
Hematuria
Candidiasis
Other functional intestinal disorders
Irritable bowel syndrome
Drug induced constipation
Fracture of lower leg, including ankle
Pain in right hip
Pathological fracture, other site, initial encounter for fracture
Hypoxemia
Vasomotor and allergic rhinitis
Abnormal tumor markers
Poisoning by, adverse effect of and underdosing of systemic antibiotics
Personal history of malignant neoplasm of prostate
Nonrheumatic mitral valve disorders
Other and unspecified diseases of blood and blood-forming organs
Gout, unspecified
Personal history of other infectious and parasitic diseases
Cerebral infarction
Encounter for therapeutic drug level monitoring
Elevated white blood cell count, unspecified
Malignant neoplasm of breast
Chronic atrial fibrillation
Poisoning by, adverse effect of and underdosing of agents primarily affecting
the gastrointestinal system
Poisoning by, adverse effect of and underdosing of drugs primarily affecting
the autonomic nervous system
Poisoning by, adverse effect of and underdosing of agents primarily acting
on smooth and skeletal muscles and the respiratory system
Poisoning by, adverse effect of and underdosing of topical agents primarily
affecting skin and mucous membrane and by ophthalmological,
otorhinorlaryngological and dental drugs
Other allergic and dietetic gastroenteritis and colitis
Presence of cardiac pacemaker
Other diseases of liver
Findings of drugs and other substances, not normally found in blood
Fracture of foot and toe, except ankle
Hereditary and idiopathic neuropathy, unspecified
Zoster [herpes zoster]
Fever presenting with conditions classified elsewhere
Family history of malignant neoplasm of breast
Lymphoid leukemia
Other neoplasms of uncertain behavior of lymphoid, hematopoietic and
related tissue
Personal history of malignant neoplasm of breast
Persons encountering health services in other specified circumstances
Respiratory failure, not elsewhere classified
Diverticular disease of intestine
Other anxiety disorders
Pain in unspecified joint
Aphagia and dysphagia
Other specified disorders of bone density and structure, unspecified site
Other abnormal findings of blood chemistry
Malignant neoplasm of unspecified site of unspecified female breast
Type 2 diabetes mellitus with diabetic chronic kidney disease
Neoplasms of unspecified behavior
Poisoning by, adverse effect of and underdosing of nonopioid analgesics,
antipyretics and antirheumatics
Poisoning by, adverse effect of and underdosing of antiepileptic, sedative-
hypnotic and antiparkinsonism drugs
Elevated blood glucose level
Encounter for other postprocedural aftercare
Chronic ischemic heart disease
Polyosteoarthritis
Complications of stem cell transplant
Other symptoms and signs involving the nervous and musculoskeletal
systems
Personal history of other malignant neoplasms of lymphoid, hematopoietic
and related tissues
Family history of malignant neoplasm of trachea, bronchus and lung
Pain in thoracic spine
Other specified disorders of bone, unspecified site
Dependence on renal dialysis
Sleep apnea, unspecified
Other specified anxiety disorders
Other diseases of digestive system
Other chest pain
Toxic gastroenteritis and colitis
Major depressive disorder, recurrent
Proteinuria, unspecified
Viral agents as the cause of diseases classified elsewhere
Syncope and collapse
Cardiomyopathy in diseases classified elsewhere
Other disorders of kidney and ureter, not elsewhere classified
Generalized edema
Other anemias
Solitary pulmonary nodule
Age-related cataract
Hypotension
Hypertensive heart disease
Acute embolism and thrombosis of unspecified deep veins of left lower
extremity
Pleural effusion, not elsewhere classified
Dysuria
Abnormal serum enzyme levels
Other forms of dyspnea
Poisoning by, adverse effect of and underdosing of other systemic anti-
infectives and antiparasitics
Viral infection of unspecified site
Other disorders of muscle
Other specified soft tissue disorders
Hyperglycemia, unspecified
Hemorrhoids and perianal venous thrombosis
Encounter for preprocedural cardiovascular examination
Psoriasis
Anemia in other chronic diseases classified elsewhere
Other conduction disorders
Personal history of (healed) other pathological fracture
Muscle weakness (generalized)
Familial hypercholesterolemia
Other symptoms and signs involving the circulatory and respiratory system
Malignant neoplasm of bronchus and lung
Collapsed vertebra, not elsewhere classified, site unspecified, initial
encounter for fracture
Other disorders of brain
Activities involving rappelling
Pain in left hip
Other disorders of skin and subcutaneous tissue, not elsewhere classified
Benign prostatic hyperplasia with lower urinary tract symptoms
Personal history of transient ischemic attack (TIA), and cerebral infarction
without residual deficits
Other primary thrombophilia
Disorders of refraction and accommodation
Other extrapyramidal and movement disorders
Old myocardial infarction
Myalgia
Multiple myeloma and malignant plasma cell neoplasms
Benign neoplasm of colon, rectum, anus and anal canal
Nicotine dependence, cigarettes, uncomplicated
Neoplastic (malignant) related fatigue
Calculus of kidney and ureter
Other iron deficiency anemias
Sleep disorders
Cramp and spasm
Osteoporosis with current pathological fracture
Myelodysplastic syndrome, unspecified
Personal history of medical treatment
Chronic sinusitis
Nonspecific elevation of levels of transaminase and lactic acid
dehydrogenase [LDH]
Estrogen receptor positive status [ER+]
Atrioventricular and left bundle-branch block
Other bacterial intestinal infections
Pain in unspecified limb
Other symptoms and signs involving the digestive system and abdomen
Other abnormal immunological findings in serum
Encounter for other specified aftercare
Malignant neoplasm of unspecified site of right female breast
Encounter for screening for infectious and parasitic diseases
Disorders of magnesium metabolism, unspecified
Plasma cell leukemia not having achieved remission
Other diseases of intestine
Chronic graft-versus-host disease
Other and unspecified noninfective gastroenteritis and colitis
Osteoarthritis of knee
Abnormal involuntary movements
Visual disturbances
Radiculopathy, lumbar region
Unspecified kidney failure
Skin changes due to chronic exposure to nonionizing radiation
Family history of malignant neoplasm of other organs or systems
Flatulence and related conditions
Prediabetes
Encounter for preprocedural laboratory examination
Cardiomegaly
Retention of urine
Adverse effect of unspecified drugs, medicaments and biological
substances, initial encounter
Complications of transplanted organs and tissue
Other and unspecified symptoms and signs involving the genitourinary
system
Presence of prosthetic heart valve

Administration of the following drugs:

bortezomib
dexamethasone
carfilzomib
daratumumab
lenalidomide
daratumumab/hyaluronidase-fihj
elotuzumab
antineoplastic-targeted/non-biologic
pomalidomide
cyclophosphamide
steroid-glucocorticoid
transplant
antineoplastic-targeted/biologic
ixazomib
antineoplastic-antineoplastic
pain agent-pain agent
solution-fluid-solution-fluid
azacitidine
doxorubicin
antiemetic-antiemetic
prednisone
isatuximab-irfc
NA-NA
etoposide
thalidomide
melphalan
fluorouracil
antineoplastic-chemotherapy
bendamustine
Cisplatin
doxorubicin pegylated liposomal
anastrozole
bone therapy agent (bta)-biphosphonate
rituximab
belantamab mafodotin-blmf
bone therapy agent (bta)-monoclonal antibody
bevacizumab
decitabine
selinexor
vincristine
leucovorin
venetoclax
leuprolide
oxaliplatin
methotrexate
gemcitabine
carboplatin
bicalutamide
pembrolizumab
letrozole
fludarabine
nivolumab
irinotecan
anti-infective-anti-infective
paclitaxel
hematological agent-hematological agent
tamoxifen
ruxolitinib
trastuzumab
capecitabine
fulvestrant
cetuximab
methoxsalen
enzalutamide
ibrutinib
docetaxel
panobinostat
levoleucovorin
antineoplastic-immunotherapy
cytarabine
blinatumomab
ado-trastuzumab emtansine
paclitaxel protein-bound
trastuzumab-anns
temozolomide
hydroxyurea
abiraterone
vismodegib
bcg vaccine
atezolizumab
rituximab-pvvr
medroxyprogesterone
hematological agent-growth factor
temsirolimus
hyperglycemic-hyperglycemic
triptorelin
cytoprotective-cytoprotective
dabrafenib
exemestane
topotecan
trametinib
imatinib
pemetrexed
mercaptopurine
vinorelbine
anticholinergic-anticholinergic
osimertinib
idecabtagene vicleucel
goserelin
melphalan flufenamide
immunosuppressive-calcineurin inhibitor
rituximab/hyaluronidase
cladribine
ponatinib
bevacizumab-awwb
tafasitamab-cxix
dasatinib
dacarbazine
rituximab-abbs
antineoplastic-antibody-conjugate
inotuzumab ozogamicin
trastuzumab-dkst
brentuximab vedotin
acalabrutinib
busulfan
obinutuzumab
ifosfamide
palbociclib
vinblastine
cabazitaxel
relugolix
nilotinib
bleomycin
immunosuppressive-immunosuppressive
ramucirumab
antineoplastic-cytoprotective
degarelix
apalutamide
cytarabine liposomal
sunitinib
pertuzumab
pazopanib
hematological agent-antianemic
proton pump inhibitor-proton pump inhibitor
tretinoin
antihyperglycemic-antihyperglycemic
antihyperglycemic-insulin/insulin analog
gout and hyperurecemia agent-gout and hyperurecemia
agent
amyloidosis agent-amyloidosis agent
antineoplastic-hormone
hormone-hormone
hormone-thyroid hormone
immunosuppressive-inosine monophosphate
dehydrogenase inhibitor

Genetic tests performed

Amplification 1q21
Deletion 13
Deletion 13q
Deletion 17p
Deletion 1p
Number of chromosomes
Other abnormality
Other Chromosome 1
Abnormalities
Ploidy
t(11; 14)
t(14; 16)
t(14; 20)
t(4; 14)
t(6; 14)
Trisomy

1. A computer-implemented method of predicting, simulating, or forecasting values of one or more specified subject-related attributes during a clinical trial, the computer-implemented method comprising:
receiving input data comprising:
a medical history of a subject, the medical history comprising values of a plurality of subject-related attributes of a subject; and
data specifying a requested output, the data comprising: the one or more specified subject-related attributes of the subject and a time frame; and
applying a trained generative machine-learning model to the received input data, the trained generative machine-learning model configured to generate output data based on the input data, the output data comprising:
respective values of the one or more specified subject-related attributes of the subject in the specified time frame
wherein the trained generative machine-learning model is a trained large language model, and,
wherein the computer-implemented method further comprises converting the received input data into converted input data having a predetermined syntax which is appropriate for input into the generative machine-learning model.

2. The computer-implemented method of claim 1, wherein:
the plurality of subject-related attributes comprises at least one longitudinal attribute.

3. The computer-implemented method of claim 2, wherein:
the plurality of subject-related attributes comprises a plurality of longitudinal attributes; and
the medical history comprises, for each longitudinal attribute of the plurality of longitudinal attributes, a plurality of values of that longitudinal attribute, each value corresponding to a measurement of that longitudinal attribute at a respective point in time.

4. The computer-implemented method of claim 1, wherein:
the trained large language model comprises one or more of: T5, LongT5, MPT, Pegasus-X, Longformer, GPT-1, GPT-2, GPT-3, GPT-3.5, GPT-4, Hyena, LLAMA, and Falcon.

5. The computer-implemented method of claim 1, wherein the generative machine-learning model has been trained using a computer-implemented method comprising:
receiving a partially trained generative machine-learning model; and
training the partially trained generative machine-learning model in a supervised manner using training data comprising a plurality of medical histories, each medical history comprising:
for a given subject, data indicative of the values of a plurality of subject-related attributes.

6. The computer-implemented method of claim 5, wherein:
the training data comprises a plurality of medical histories, each medical history comprising:
for a given subject, data indicative of the values of a plurality of subject-related attributes, the plurality of subject-related attributes comprising a plurality of longitudinal attributes, and the training data comprising, for each attribute of the plurality of longitudinal attributes, a plurality of values of that longitudinal attribute, each value corresponding to a measurement of that longitudinal attribute at a respective time.

7. The computer-implemented method of claim 5, wherein:
training the generative machine-learning further comprises:
receiving raw training data; and
converting the raw training data to converted training data having a predetermined syntax which is appropriate for input into the generative machine-learning model.

8. The computer-implemented method of claim 7, wherein:
the converted training data is in a JavaScript Object Notation (JSON) format, the JSON comprising a first portion and a second portion, the first portion comprising data defining values of longitudinal attributes and the second portion comprising data defining values of static attributes; and
the converted training data comprises dates expressed in relative terms to an earliest date.

9. The computer-implemented method of claim 1, wherein:
the converted input data is in a JavaScript Object Notation (JSON) format, the JSON comprising a first portion, a second portion, and a third portion, the first portion comprising data defining values of longitudinal attributes, the second portion comprising data defining values of static attributes, and the third portion comprising the data specifying the requested output; and
the converted input data comprises dates expressed in relative terms to an earliest date.

10. The computer-implemented method of claim 1, wherein:
the data specifying a requested output may further comprise data identifying a therapeutic intervention, such that the generative machine-learning model is configured to generate an output indicative of an effect of the therapeutic intervention on the subject.

11. The computer-implemented method of claim 10, wherein:
the training data comprises a plurality of medical histories relating to subjects who have been treated using the therapeutic intervention, the medical histories comprising data indicating that the subjects have been treated using the therapeutic intervention.

12. The computer-implemented method of claim 1, further comprising, after the output data has been generated:
i. generating modified input data by combining the input data with the output data;
ii. applying the trained generative machine-learning model to the modified input data to generate updated output data; and
iii. repeating steps (i) and (ii) until an end condition is met.

13. A computer-implemented method of determining an efficacy and/or safety of a trial therapeutic intervention in a clinical trial, the computer-implemented method comprising:
receiving electronic data comprising results of a clinical trial relating to a trial therapeutic intervention;
receiving control data, the control data generated by:
receiving input data comprising:
a medical history of a subject, the medical history comprising values of a plurality of subject-related attributes of a subject; and
data specifying a requested output, the data comprising one or more specified subject-related attributes of the subject and a time frame; and
applying a trained generative machine-learning model to the received input data, the trained generative machine-learning model configured to generate control data based on the input data, the control data comprising:
respective values of the one or more specified subject-related attributes of the subject in the specified time frame
wherein the trained generative machine-learning model is a trained large language model, wherein the computer-implemented method further comprises converting the received input data into converted input data having a predetermined syntax which is appropriate for input into the generative machine-learning model; and
determining an efficacy and/or safety of the trial therapeutic intervention based on a comparison of the electronic data comprising the results of the clinical trial with the control data comprising the generated data.

14. The computer-implemented method of claim 13, wherein:
determining an efficacy and/or safety comprises determining a value of an efficacy and/or safety metric indicative of the trial therapeutic intervention; and
selecting the trial therapeutic intervention for further investigation based on the value of the efficacy and/or safety metric.
